(19) United States
(12) Patent Application Publication
(10) Pub. No.: US 2022/0088221 A1
(43) Pub. Date: Mar. 24, 2022

(54) SELF-ASSEMBLED PEPTIDE NANOPARTICLE AND USE THEREOF

(71) Applicant: THE METHODIST HOSPITAL, Houston, TX (US)

(72) Inventors: Rongfu WANG, Temple City, CA (US); Yicheng WANG, Temple City, CA (US); Motao ZHU, Pearland, TX (US); Ruifang ZHAO, Beijing (CN)

(21) Appl. No.: 17/422,309

(22) PCT Filed: Jan. 13, 2020
(86) PCT No.: PCT/US20/13417

§ 371 Date: Jul. 12, 2021

Related U.S. Application Data
(60) Provisional application No. 62/791,795, filed on Jan. 12, 2019.

(51) Int. Cl. A61K 47/69; A61K 47/64; A61K 39/00; A61P 35/00; A61K 45/06

(52) U.S. Cl. CPC A61K 47/6929; A61K 47/6455; A61K 39/001156; A61K 2039/55561; A61P 35/00; A61K 45/06; A61K 2039/55555; A61K 39/001188

(57) ABSTRACT

Disclosed are self-assembling nanoparticle compositions that comprise a plurality of cationic cell penetrating peptides, each covalently linked to a hydrophobic therapeutic molecule (e.g., an antigenic peptide, mRNA, siRNA, DNA, or the like), and optionally, non-covalently bound to at least one TLR (Toll-like receptor) ligand. Also disclosed are methods for use of the nanoparticle compositions in the treatment, prophylaxis, and/or the amelioration of one or more symptoms of a mammalian disease, including, without limitation, cancer, infection, inflammation, and related diseases and abnormal conditions.

### CROSS-REFERENCE TO RELATED APPLICATIONS

The present application claims priority to United States Provisional Patent Application 62/791,795, filed Jan. 12, 2019, the contents of which is specifically incorporated herein in its entirety by express reference thereto.

### STATEMENT REGARDING FEDERALLY SPONSORED RESEARCH OR DEVELOPMENT

Not Applicable.

### NAMES OF THE PARTIES TO A JOINT RESEARCH AGREEMENT

Not Applicable.

### BACKGROUND OF THE INVENTION

### Field of the Invention

The present disclosure relates to a nanoparticle comprising a multitude of cationic cell penetrating peptides (CPPs) each covalently linked to a hydrophobic therapeutic peptide, e.g., an antigen peptide, and optionally at least one or multiple TLR (Toll-like receptor) ligands non-covalently bound to the CPP-linked therapeutic peptides. This amphipathic property of the CPP-linked therapeutic peptides has self-assembling ability to form nanoparticles with negative charged nucleic acids (such as CpG, poly (I:C), mRNA, siRNA and DNA) and hydrophobic MPLA under neutral condition (pH=7.0) but disrupted under acidic conditions (pH<5). The resulting self-assembled nanoparticles containing CPP-linked therapeutic peptide and TLR ligands or mRNA allow co-delivery into antigen-presenting cells (APCs) for efficient presentation for T cell activation, leading to the generation of potent immunity against cancer and other diseases. Thus, the present disclosure also provides a method for treatment and/or prophylaxis of a cancer, including various tumors, or an infectious disease by employing CPP-T-cell peptide/TLR ligand assembled nanoparticles.

### Description of Related Art

1. Cancer Immunotherapy

Cancer is a leading cause of deaths in the United States and worldwide, posing a major public health problem. Cancer immunotherapy has been a promising approach to cancer therapy (Di Lorenzo et al., 2011; Lesterhuis et al., 2011; Rosenberg, 2011; Wang and Wang, 2017). Several immunotherapy-based checkpoint blockade drugs, such as the cytotoxic T-lymphocyte-associated protein 4 (CTLA-4) monoclonal antibody (Ab), ipilimumab (Yervoy), the programmed cell death (PD)-1 Ab, pembrolizumab (Keytruda) have been approved by the U.S. Food and Drug Administration (FDA) for treatment of many types of cancer (Bagcchi, 2014; Hodi, 2010; Kantoff et al., 2010; Bender, 2017). Furthermore, cell-based immunotherapy using T cell receptor (TCR) or chimeric antigen receptor (CAR)-engineered T cells has shown promising clinical responses in blood cancers, such as leukemia and lymphoma.

Despite these rapid progresses, the majority of cancer patients generally do not respond to checkpoint blockade therapy. For example, approximately 20% of lung cancer patients respond to immune checkpoint therapy. Only 13-18% of breast and prostate cancer patients respond to immune checkpoint therapy (Nanda et al., 2016; Kwon et al., 2014). CAR-T cell immunotherapy technology works well in the blood cancer (Sadelain et al., 2017; Johnson and June, 2017), but it does not work well in solid cancer, probably due to immunosuppression in the tumor microenvironment. Recent studies show that immune checkpoint blockade therapy relies on the presence of tumor-reactive T cells in the tumor tissues for their clinical effectiveness, and are correlated with tumor-infiltrating T cells, PD-L1 expression and mutational loads (Sharma et al., 2017). Cancer patients, whose tumor tissues lack tumor-infiltrating antigen-specific T cells, generally fail to respond to immune checkpoint therapy. To overcome these issues, cancer vaccines could increase tumor-specific T cells for tumor control. Alternative strategy is to adoptively transfer tumor-specific T cells that are either derived from cancer patients or engineered to express tumor antigen-specific TCR or CAR on T cells.

Immunotherapy with cancer vaccines offers the potential for high tumor-specific cytotoxicity, and thus is a very attractive approach for cancer treatment. In fact, the first therapeutic cancer vaccine (Sipuleu-cel-T; PROVENGE©, Dendreon) was approved by FDA in 2010 for the treatment of metastatic prostate cancer (Kantoff et al., 2010). However, cancer vaccines generally have only met with limited clinical success. Even for the FDA-approved Sipuleu-cel-T (PROVENGE©) vaccine, there is no evident clinical response, but with extension of the patient survival to 4.1 months compared with the control group. Vaccination with antigenic peptides or dendritic cells (DCs) pulsed with antigenic peptides can generate antitumor immunity but fail to generate sufficient immune response to obtain significant clinical benefit in several types of cancer being tested (Melero et al., 2014; Rosenberg et al., 2004). DC/peptides or protein vaccines alone may not be powerful enough to generate potent, long-lasting antitumor responses (Rosenberg et al., 2004).

Previous studies have shown that intracellular delivery of cancer antigenic peptides such as tyrosinase-related protin-2 (TRP-2) into DCs through a cell-penetrating peptide (CPP) covalently linkage enhances antigen-specific T cell response and antitumor immunity against cancer, mainly due to the prolonged antigen presentation time of DCs to T cells (Wang and Wang, 2002; Wang et al., 2002). Based on these preclinical studies, clinical studies using TAT-NY-ESO-1 peptide were initiated and found that such peptide vaccines are safe and can induce antigen-specific T cell response in 6 of 9 prostate cancer patients evaluated, which correlated with the increased PSA double time in the vaccinated patients (Sonpavde et al., 2014). However, overall immune response is too weak and transient to induce cancer regression. Therefore, novel strategies are urgently needed to develop more potent vaccines against cancer and other diseases.

Toll-like receptors (TLRs) have recently emerged as a critical component of the innate immune system, wherein they detect microbial infection and activate DC maturation programs for the induction of adaptive immune responses (Iwasaki and Medzhitov, 2004; Akira and Takeda; 2016). Triggering innate immune receptors, such as TLRs, Nod-like receptors (NLRs), and RIG-like receptors (RLRs), in DCs, with their corresponding ligands, activates nuclear factor-κB (NFκB), type I interferon (IFN), and inflammatory responses. These signaling pathways produce proinflammatory cytokines and induce strong innate and adaptive immune responses. Administration of an antigen, together with TLR ligands, might increase the immunogenicity of the antigen and increase the ability of DCs to prime T cell responses (Blander and Medzhitov; 2006; Blander and Medzhitov; 2006). Loading DCs with antigenic peptides, together with TLR ligands, may be efficacious in generating strong T cell responses using peptides and TLR ligands (Palucka and Banchereau, 2013).

Using nanotechnology such as multi-stage vectors (MSV), more antigens could be loaded into nanoliposomes or nanoparticles and a stronger antitumor immunity could be generated against breast cancer as compared with conventional DC vaccines (Xia et al., 2015). In melanoma, it was found that cancer antigenic peptide TRP-2 must be co-loaded with TLR ligands (CpG and MPLA) into MSV, and then uptake by the same DCs as vaccines (Zhu et al., 2018). Vaccination of a mixture of MSV/TRP-2-loaded DCs with MSVTLR ligands-loaded DCs did not generate potent antitumor immunity, suggesting that co-delivery of peptide and TLR ligands is critically important (Zhu et al., 2018). However, despite the advance made with MSV technology, DC/MSV-based vaccination could extend mouse survival for a limited time (10 days), further suggesting that DC/MSV-based vaccination only delay tumor growth, but could not generate sufficient antitumor immunity to complete eliminate tumor cells.

Based on these studies, it was reasoned that the current vaccine strategies failed to generate sufficient immunity to completely eliminate cancer cells. Alternatively, immunosuppression in the tumor microenvironment inhibits antitumor immunity induced by peptide vaccines. To understand why DCs loaded with CPP-linked therapeutic peptide fails to elicit antitumor immunity to eradicate cancer, it was found that CPP-linked therapeutic peptide facilitates the intracellular delivery of antigenic peptide into DCs. Similarly, CPPs have been used to deliver different cargos, including proteins, DNAs, siRNA and mRNA into cells, into target cells. However, it was found that CPP TAT-NY-ESO-1 peptide was difficult for generating a stable emulsion with the vaccine adjuvant, Montanide ISA-51. The emulsion drops of TAT-NY-ESO-1 and Montanide ISA-51 were unstable and diffused within a short of time in the water, which might influence vaccine efficacy. This unstable property of TAT-NY-ESO-1 and Montanide ISA-51 prompted the inventors to further investigate how to overcome this problem. One potential problem is that the positive charges (hydrophilic) of CPP (i.e., TAT) might disrupt the emulsion of CPP-NY-ESO-1 and Montanide ISA-51.

A strong vaccine must contain an innate immune signaling component. A recent study by the inventors and their collaborators showed that co-delivery of antigenic peptides and TLR ligands into the same DCs is essential for generating potent and effective immune response (Zhu et al., 2018). Although MSV-based approach improves the co-delivery of antigenic peptides and nucleic acid-based TLR ligands into the same DCs, thus enhancing antitumor immunity. However, this approach did not solve the fundamental problem, i.e., antigenic peptide and nucleic acid-based TLR3 and TLR9 ligands do not form a complex due to hydrophobic property of peptides and negative charged nucleic acids.

In view of the above, there is a need for improved delivery methods for cancer therapeutic molecules, including T cell epitopes, B cell epitopes, therapeutic nucleic acid molecules and adjuvants.

2. Self-Assembled, Peptide-Based Nanostructures

Molecular self-assembly is the spontaneous formation of ordered structures, and it occurs under thermodynamic and kinetic conditions because of specific and local molecular interactions. Hydrogen bonding, hydrophobic interactions, electrostatic interactions, and van der Waals forces combine to maintain molecules at a stable low-energy state. Self-association to form hierarchical structures at both the nano- and/or micro-scales occurs to achieve these energy minima (Han et al., 2010).

Self-assembly occurs spontaneously in nature during protein folding, DNA double-helix formation, and the formation of cell membranes (Korolkov et al., 2013). Self-assembling nanostructures fabricated from natural biomolecular building blocks such as amino acids are highly preferable to their synthetic self-assembled monolayer (SAMs) alternatives (Tayebe Zohrabi et al., 2015) due to their biocompatibility and ease of “bottom-up” fabrication (Yan et al., 2010).

3. Cell Penetrating Peptides

Cell Penetrating Peptides (CPPs) are generally described as short peptides of 8-30 amino acids, capable of penetrating biological membranes to trigger the movement of various biomolecules across cell membranes into the cytoplasm and to improve their intracellular routing, thereby facilitating interactions with the target (See e.g., U.S. Pat. No. 9,598,465). CPPs are either derived from proteins or from chimeric sequences, usually amphipathic and possess a net positive charge (Morris, et al., 2008; Hansen et al., 2008; Heitz et al., 2009). Several CPPs have been identified, from proteins, including the Tat protein of human immunodeficiency virus (HIV) (Frankel and Pabo, 1988), the VP22 protein of herpes simplex virus (Elliott and O'Hare; 1997; Phelan et al., 1998), and the fibroblast growth factor (Lin et al., 1995; Rojas et al., 1998). The Tat peptide and membrane-translocating sequence (MTS) have been used to transduce proteins into cells both in vitro and in vivo (Farwell et al., 1994; Kim et al., 1997; Schwarz et al., 1999; Lindgren et al., 2000).

CPPs can be subdivided into two main classes, the first requiring chemical linkage with the cargo, and the second involving the formation of stable, non-covalent complexes. CPPs have been used for the delivery of a large panel of cargos (plasmid DNA, oligonucleotide, siRNA, PNA, protein, peptide, liposome, nanoparticle) into a wide variety of cell types and in vivo models (Morris et al., 2008; Beggars and Sagan, 2013; Huang et al., 2015; Marcus et al., 2016; Gungor et al., 2014). In these cases, CPPs mainly carry the cargo into cells through their membrane-translocating ability (FIG. 1). In these applications, there is no therapeutic T cell epitope that covalently linked to CPPs. Thus, CPPs do not the amphipathic property and are different from CPP-T cell peptide, which have an amphipathic property to self-assemble into nanoparticles with negatively charged molecules.

### BRIEF DESCRIPTION OF THE INVENTION

The present invention overcomes these and other limitations inherent in the prior art by providing novel vaccines with a targeted delivery system. Disclosed are self-assembling nanoparticles comprised of populations of cationic cell penetrating peptides (CPPs) linked to one or more hydrophobic therapeutic peptide ligands, including a TLR (toll-like receptor) and antigen peptides. The amphipathic property of the resulting nanoparticles (i.e., having both hydrophilic and hydrophobic moieties) additionally facilitates the inclusion one or more therapeutic mRNAs, siRNAs, and/or DNA molecules. The resulting particles are self-assembling at neutral pH, and can be delivered into antigen-presenting cells (APCs) such as dendritic cells for presentation to T-cells, leading to activation of the immune system, or they can be delivered directly as a vaccine.

In a particular embodiment, the inventors have demonstrated that cationic CPPs each covalently linked to a certain therapeutic peptide, e.g., an antigen peptide, which preferably is hydrophobic, can form a tight and small-sized (50-100 nm) self-assembled nanoparticle, and can be used to achieve efficient intracellular delivery of the therapeutic peptides. Other components, such as negatively charged molecules (DNA, dsRNA, siRNA, or mRNA), can be included in the nanoparticle, for facilitating nanoparticle formation and nanoparticle delivery across cell membranes (FIG. 2). The nanoparticle comprises: (i) a corona comprising the CPPs with positively charged peptide that is covalently linked to a therapeutic peptide with preferred hydrophobic property, and (ii) negatively charged molecules (DNA, dsRNA, siRNA, or mRNA) plus hydrophobic molecules such as MPLA. Based on the electric charges and hydrophobic properties, we designed and developed a novel technology of self-assembled CPP-T-cell peptide nanoparticles with TLR ligands [CpG and MPLA, CM for short; CpG and MPLA and poly (I:C), CMI for short], as schematically presented in FIG. 2A and FIG. 2B. Amphiphobic or amphipathic CPP-therapeutic peptides, consisting of CPP such as TAT with positively charged peptide and covalently linked to a therapeutic peptide such as NY-ESO-1 [SLLMWITQCFLPV (SEQ ID NO:1)] and TRP-2 [SYVDFFVWL (SEQ ID NO:2)] (generally hydrophobic), form nanoparticles with negatively charged CpG and/or poly (I:C) through electric interactions, while with MPLA through hydrophobicity inside the particles.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The following drawings form part of the present specification and are included to demonstrate certain aspects of the present invention. For promoting an understanding of the principles of the invention, reference will now be made to the embodiments, or examples, illustrated in the drawings and specific language will be used to describe the same. It will, nevertheless be understood that no limitation of the scope of the invention is thereby intended. Any alterations and further modifications in the described embodiments, and any further applications of the principles of the invention as described herein are contemplated as would normally occur to one of ordinary skill in the art to which the invention relates.

The invention may be better understood by reference to the following description taken in conjunction with the accompanying drawings, in which like reference numerals identify like elements, and in which:

FIG. 1 illustrates that CPP functions a carrier to deliver cargo into cells;

FIG. 2A and FIG. 2B show cationic CPP-T cell epitope peptide has an amphopathetic property to form a nanoparticles and delivery into endosomes. FIG. 2A: Cationic CPP (positive charges) is covalently linked with a T cell epitope peptide (hydrophobic) to have an amphopatheic property. CpG and poly(I:C) are negatively charged, while MPLA is hydrophobic. CPP-T cell peptide forms a nanoparticle with CpG and/or poly(I:C) through electric interactions, while through hydrophobic interaction with MPLA inside particles. FIG. 2B: CPP-peptide/TLR ligands nanoparticles are uptaken by DCs or macrophages and delivery into endosomes, where CPP-peptide/TLR ligand particles are disrupted at pH 4.0. CPP-peptides are processed and presented by MHC class I or II molecules to T cells, while TLR ligands binds to TLR3, TLR4 and TLR9 to trigger innate immune response and cytokine production, thus enhancing the efficiency and quality of T cell responses;

FIG. 3A-1, FIG. 3A-2, FIG. 3B-1, FIG. 3B-2, FIG. 3C-1 and FIG. 3C-2 show self-assembled nanoparticles of TAT-TRP2 with CpG and MPLA (CM for short). TRP2 itself could not form a particle with CpG and MPLA. AFM analysis indicates the size of cross section of the nanoparticle;

FIG. 4 shows DLS measurement for size distributions of self-assembled nanoparticles of TAT-TRP2 with TLR ligands at various ratios. The combinations of TAT-TRP2 and TLR ligands (different ratios) are listed in Table 3. Grey bars indicate unstable/polydispersed complex with large PDI (PDI>0.5);

FIG. 5 shows the Zeta potential of TAT-TRP2-CM complex constituted of TAT-TRP2, CpG and MPLA at various nitrogen (+) over phosphate (−) (N/P) ratios;

FIG. 6A and FIG. 6B show the characterization of TAT-TRP2-CM complex. DLS measurement for the sizes of TAT-TRP2-CM complex in H₂O (FIG. 6A). Zeta potential of TAT-TRP2 (or TRP2^{TAT}), CpG, MPLA and TAT-TRP2-CM complex in H₂O (FIG. 6B). PDI, polydispersity index;

FIG. 7A, FIG. 7B, FIG. 7C, FIG. 7D, FIG. 7E, and FIG. 7F show nanoparticles of TAT-TRP2 and TAT-ESO-1 with CpG and MPLA (TAT-TRP2-CM, TAT-ESO-1-CM), and of TAT-TRP2 and TAT-ESO-1 with CpG, MPLA and poly (I:C) (TAT-TRP2-CMI and TAT-ESO-1-CMI). Schematic presentation of TAT-TRP2 and TAT-ESO-1 nanoparticles (FIG. 7A). Nanoparticles sizes of TAT-TRP2 and TAT-ESO-1 with CM (FIG. 7B) or CMI (FIG. 7C and FIG. 7D). FIG. 7E and FIG. 7F: Zeta potential of TAT-TRP2-CM, TAT-TRP2-CMI, TAT-ESO-1-CM and TAT-ESO-1-CMI;

FIG. 8A, FIG. 8B, and FIG. 8C show the characterization of TAT-TRP2-CM complex at different pH values. (FIG. 8A and FIG. 8B) DLS measurement for the sizes of TAT-TRP2-CM complex in pH 7.0 (FIG. 8A) and pH 4.0 (FIG. 8C) potassium phosphate buffer. (FIG. 8C) Zeta potential of TAT-TRP2 (or TRP2^{TAT}), CpG, MPLA and TAT-TRP2-CM complex in pH 7.0 and pH 4.0 potassium phosphate buffer. PDI, polydispersity index;

FIG. 9A, FIG. 9B, FIG. 9C, and FIG. 9D show the assembly and nanoparticles of TAT-TRP2/TLR and TAT-ESO-1/TLR are pH-dependent. FIG. 9A shows DLS measurement for TAT-TRP2-CM complex size change in pH 4-7 potassium phosphate buffers. FIG. 9B is a schematic illustration for TAT-TRP2 or TAT-TRP2-CM complex uptake and pH-dependent complex dissembling process in DCs. FIG. 9C and FIG. 9D show DLS measurement for TAT-TRP2-CM (FIG. 9C), TAT-ESO-1-CM TAT-TRP2-CMI (FIG. 9D), and TAT-ESO-1-CMI complex size change in pH 7 and pH 4 potassium phosphate buffers;

FIG. 10 shows combinations of TLR ligands for stimulating innate immune responses and cytokine production. Bone-marrow-derived DCs were isolated and then treated with different TLR ligands (single alone), double or triple combinations. Cytokine (TNF-α, IL-6, IFN-α and IFN-β) production in cell supernatants was determined by ELISA. The poly (I:C)/CpG, CpG/MPLA double combinations, and CpG/poly(I:C)/MPLA triple combination were stronger than other groups in triggering innate immune cytokine production;

FIG. 11 shows lung metastasis model of B16 tumor in C57BL/6 mice vaccine with DC/TAT-TRP2-CM and DC/TRP2-CM. B16 tumor cells (0.2×10⁶ cells/mouse) were injected (i.v.) into C57BL/C mice at day 0, and vaccine with DC/TAT-TRP2-CM, DC/TRP2-CM or DC/beta-gal-CM (5×10⁶ cells/mouse) at day 5. Mice were sacrificed at day 18. The number of lung metastases were counted;

FIG. 12A, FIG. 12B, and FIG. 12C show lung metastasis and survival of B16-bearing C57BL/6 mice after various vaccinations. FIG. 12A shows the tumor model and vaccine schedule. In FIG. 12B, B16 tumor cells (0.2×10⁶ cells/mouse) were injected (i.v.) into C57BL/C mice at day 0, and 5 different groups (5×10⁶ cells/mouse) with vaccine at day 5. All mice were sacrificed at day 18. The number of lung metastases were counted. FIG. 12C shows B16 injection and vaccines were the same as in FIG. 12B. B16-bearing mice vaccinated with different groups were monitored for their survival for 55 days. Error bars represent standard deviation. *p<0.05, **p<0.01, ***p<0.001;

FIG. 13A and FIG. 13B show DC/TAT-ESO-CM and TAT-ESO-CMI vaccines produce strong antitumor immunity. HLA-A2 Tg mice were injected with RM1/A2-ESO-1 tumor cells at day 0. Tumor-bearing mice were treated with vaccines (DC/control, DC/TAT-ESO-CM or DC/TAT-ESO-CMI). Tumor growth was monitored every two days. FIG. 13A shows tumor sizes on day 15. FIG. 13B shows the tumor growth curves. P value is indicated among different groups;

FIG. 14A and FIG. 14B show the T cell responses in mice vaccinated with DC/control, DC/TAT-ESO-CM or DC/TAT-ESO-CMI (FIG. 14A). FIG. 14B shows the % IFN-γ in CD8⁺ cT-cells;

FIG. 15 shows the marked inhibition of breast cancer growth after DC/TAT-ESO-CM vaccination. HLA-A2 Tg mice were injected with E0771/A2-ESO-1 tumor cells at day 0. Tumor-bearing mice were treated with DC/control or DC/TAT-ESO-CM. Tumor growth was monitored every two days. Vaccination with DC/TAT-ESO-CM markedly inhibited breast cancer growth;

FIG. 16A, FIG. 16B, and FIG. 16C show direct immunization with TAT-ESO-CMI generated potent and therapeutic antitumor immunity, compared with DC/TAT-ESO-CMI vaccination. In FIG. 16A, HLA-A2 Tg mice were injected with RM1/A2-ESO tumor cells at day 0, followed by three injections of TAT-ESO-CMI at days 10, 13 and 18 or DC/TAT-ESO-CMI vaccination at day 10. Tumor growth was monitored every two days. In FIG. 16B, tumor sizes are shown with each group. In FIG. 16C, tumor growth of three groups after vaccination was recorded. P-values are shown for significance among groups;

FIG. 17A, FIG. 17B, FIG. 17C, and FIG. 17D show CT83 expression in breast cancer samples and cell lines. FIG. 17A shows the RF-PCR analysis of CT83 expression breast cancer cell lines. FIG. 17B shows CT83 expression in breast cancer samples using RT-PCR analysis. NY-ESO-1 served as a positive control. FIG. 17C is a Western blot analysis of breast cancer cell lines using an anti-CT83 antibody. Antibody-staining of normal and breast cancer tissues for CT83 expression is shown in FIG. 17D.

FIG. 18A and FIG. 18B show CT83 expression in lung cancer samples and cell lines. FIG. 18A shows RF-PCR analysis of CT83 expression lung cancer cell lines and cancer samples. FIG. 18B shows Western blot analysis of CT83 expression in lung cancer cell lines using an anti-CT83 antibody. MDA-468 served as a positive control;

FIG. 19A, FIG. 19B, FIG. 19C, and FIG. 19D show the generation of CD83-specific T cells using self-assembled TAT-CT83 peptide nanoparticles with CMI. FIG. 19A shows the intracellular staining of IFN-γ release from splenocytes of TAT-CT83-CMI immunized mice. FIG. 19B shows IFN-γ release assay by ELISA. FIG. 19C shows T cell response to CT83 peptides after one cycle of culture. FIG. 19D shows the establishment of CT83-A2 restricted peptide T cell clones;

FIG. 20A, FIG. 20B, FIG. 20C, and FIG. 20D show TAT-CT83-CMI vaccine generates potent anti-tumor immunity against E0771-A2-CT83 breast cancer. (FIG. 20A) Scheme for the establishment of E0771-A2-CT83 breast cancer model in HLA-A2 transgenic mice and vaccination schedule. (FIG. 20B) Images of E0771-A2-CT83 breast tumor after 2 times immunization with indicated vaccine formulations alone or along with anti-PD1 blockade therapy (10 mg/kg BW, i.p). (FIG. 20C) The weights of tumors from mice vaccinated with indicated formulations with or without anti-PD1 antibody were isolated and measured on day 16. (FIG. 20D) Immunohistochemistry staining of CD3⁺ T cell infiltration in tumor site with or without TAT-CT83-CMI vaccination;

FIG. 21A, FIG. 21B, FIG. 21C, and FIG. 21D show vaccination of TAT-ESO-CMI generates potent therapeutic immunity against breast cancer. (FIG. 21A) A schematic presentation of vaccine experimental design. (FIG. 21B) Tumor growth is shown in each group in HLA-A2 Tg mice. (FIG. 21C) The images of mice and tumor sizes. (FIG. 21D) Vaccination with TAT-ESO-CMI without DCs markedly inhibited tumor growth. *P<0.05, **P<0.01;

FIG. 22A, FIG. 22B and FIG. 22C show TAT-TRP2-CMI vaccine alone or in combination with anti-PD-1 therapy. (FIG. 22A and FIG. 22B) Lung images and the number of lung metastasis of mice received vaccination of TAT-TRP-2/CMI alone or in combination with anti-PD-1 treatment. (FIG. 22C) Mouse survival after TAT-TRP-2/CMI vaccination alone or in combination with anti-PD-1 therapy;

FIG. 23A and FIG. 23B show TAT-ESO-CMI vaccine alone or in combination with anti-PD-1 therapy. (FIG. 23A) tumor images of RM1/A2-ESO tumor cells in HLA-A2-Tg mice received vaccination of TAT-ESO-CMI alone or in combination with anti-PD-1 treatment, compared with control group. (FIG. 23B) Tumor growth curves after TAT-ESO-CMI vaccination alone or in combination with anti-PD-1 therapy, compared with control group;

FIG. 24A and FIG. 24B show TCR-T cell transfer followed by SAPNANO vaccine expanded tumor-infiltrating T cells and markedly inhibited tumor growth. (FIG. 24A) Tumor growth among different treatment groups. *P value<0.05. (FIG. 24B) Increased percentage of tumor-infiltrating A2-ESO TCR-T cells after A2-ESO TCR-T cells alone or in combination of TAT-ESO-CMI vaccines. A2-ESO TCR-T cells were detected using anti-TCR human Vβ13 antibody dating on anti-CD3 positive T cells. TAT-ESO-CMI vaccine induced endogenous T cells could not be detected with anti-TCR human Vβ13 antibody; and

FIG. 25A, FIG. 25B, FIG. 25C, FIG. 25D, and FIG. 25E show combination of NY-ESO TCR-T therapy and TAT-ESO-CMI vaccination generated strong anti-tumor response in humanized NSG mice model. (FIG. 25A) Schematic diagram of animal experiment. 3˜4-week old NSG mice were humanized by intravenous injecting 1×10⁷ of human PBMC to reconstitute human immune system 3˜4 weeks before tumor incubation. HLA-A2 and NY-ESO positive human breast cancer cells (MDA-MB-231-A2-ESO) were subcutaneously injected to the fat pad of humanized NSG mice (1 million/mouse). Tumor bearing mice were treated by NY-ESO TCR-T cells at day 5. Three doses of human IL2 (50000 IU) and 4 doses of TAT-ESO-CMI vaccine were administrated through i.v. injection. Mice were sacrificed at day 30. (FIG. 25B) Human lymphocytes were detected by FACS in NSG mice after 3-weeks of humanization. (FIG. 25C) Tumor growth was monitored after treatment. Data represent as Mean±SEM. PBS control group (N=4), other groups (N=5), two-way ANOVA test was used for statistics analysis. *p<0.05, **p<0.01. (FIG. 25D and FIG. 25E) Tumor weight was imaged and weighted after isolation from mice. (Mean±SEM, T test was used for statistics analysis. *p<0.05, **p<0.01, ***p<0.001)

### BRIEF DESCRIPTION OF THE SEQUENCES

SEQ ID NO:1 is an exemplary therapeutic NY-ESO-1-specific peptide for use in accordance with one aspect of the present disclosure;

SEQ ID NO:2 is an exemplary therapeutic TRP-2-specific peptide for use in accordance with one aspect of the present disclosure;

SEQ ID NO:3 is an exemplary HIV Tat 47-57-specific cell penetrating peptide sequence for use in accordance with one aspect of the present disclosure;

SEQ ID NO:4 is an exemplary TAT-PTD-4-specific cell penetrating peptide sequence for use in accordance with one aspect of the present disclosure;

SEQ ID NO:5 is an exemplary TAT-PTD-5-specific cell penetrating peptide sequence for use in accordance with one aspect of the present disclosure;

SEQ ID NO:6 is an exemplary DPV3-specific cell penetrating peptide sequence for use in accordance with one aspect of the present disclosure;

SEQ ID NO:7 is an exemplary DPV6-specific cell penetrating peptide sequence for use in accordance with one aspect of the present disclosure;

SEQ ID NO:8 is an exemplary DPV7-specific cell penetrating peptide sequence for use in accordance with one aspect of the present disclosure;

SEQ ID NO:9 is an exemplary nine-residue poly-arginine cell penetrating peptide sequence for use in accordance with one aspect of the present disclosure;

SEQ ID NO:10 is an exemplary nine-residue poly-lysine cell penetrating peptide sequence for use in accordance with one aspect of the present disclosure;

SEQ ID NO:11 is an exemplary FHV coat-specific cell penetrating peptide sequence for use in accordance with one aspect of the present disclosure;

SEQ ID NO:12 is an exemplary Signal-peptide II-specific cell penetrating peptide sequence for use in accordance with one aspect of the present disclosure;

SEQ ID NO:13 is an exemplary amphiphilic model peptide-specific cell penetrating peptide sequence for use in accordance with one aspect of the present disclosure;

SEQ ID NO:14 is an exemplary HSV VP22-specific cell penetrating peptide sequence for use in accordance with one aspect of the present disclosure;

SEQ ID NO:15 is an exemplary peptide carrier-specific cell penetrating peptide sequence for use in accordance with one aspect of the present disclosure;

SEQ ID NO:16 is an exemplary CL22-specific cell penetrating peptide sequence for use in accordance with one aspect of the present disclosure;

SEQ ID NO:17 is an exemplary TRP-2specific peptide sequence for use in accordance with one aspect of the present disclosure;

SEQ ID NO:18 is an exemplary TAT-linked peptide for use in accordance with one aspect of the present disclosure;

SEQ ID NO:19 is an exemplary TAT-linked peptide for use in accordance with one aspect of the present disclosure;

SEQ ID NO:20 is an exemplary TAT-linked peptide for use in accordance with one aspect of the present disclosure;

SEQ ID NO:21 is an exemplary TAT-linked peptide for use in accordance with one aspect of the present disclosure;

SEQ ID NO:22 is an exemplary TAT-linked peptide for use in accordance with one aspect of the present disclosure;

SEQ ID NO:23 is an exemplary TAT-linked peptide for use in accordance with one aspect of the present disclosure; and

SEQ ID NO:24 is an exemplary TAT-linked peptide for use in accordance with one aspect of the present disclosure.

SEQ ID NO:25 is an exemplary TAT-linked peptide for use in accordance with one aspect of the present disclosure;

SEQ ID NO:26 is an exemplary TAT-linked peptide for use in accordance with one aspect of the present disclosure;

SEQ ID NO:27 is an exemplary TAT-linked peptide for use in accordance with one aspect of the present disclosure; and

SEQ ID NO:28 is an exemplary TAT-linked peptide for use in accordance with one aspect of the present disclosure.

### DESCRIPTION OF ILLUSTRATIVE EMBODIMENTS

Illustrative embodiments of the invention are described below. In the interest of clarity, not all features of an actual implementation are described in this specification. It will of course be appreciated that in the development of any such actual embodiment, numerous implementation-specific decisions must be made to achieve the developers' specific goals, such as compliance with system-related and business-related constraints, which will vary from one implementation to another. Moreover, it will be appreciated that such a development effort might be complex and time-consuming but would be a routine undertaking for those of ordinary skill in the art having the benefit of this disclosure.

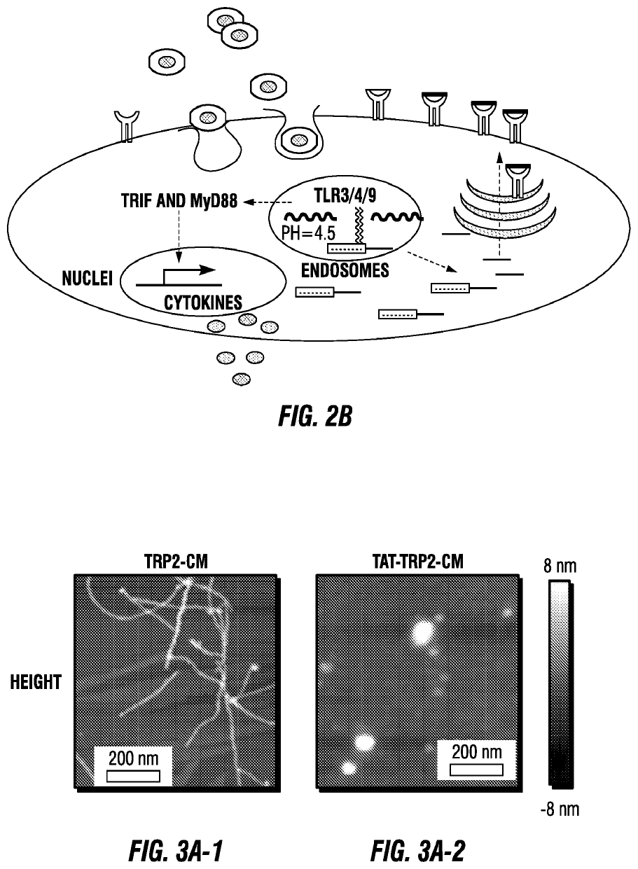

Therefore, in a first aspect, the present invention provides a nanoparticle comprising a multitude of cationic CPPs each covalently linked to a hydrophobic therapeutic peptide, which is self-assembled under neutral condition, e.g., at pH 7.0, and disassociated under acidic condition, e.g., at pH 4.5 (FIG. 2A and FIG. 2B).

In some embodiments, the cationic CPP is selected from the group consisting of TAT, TAT-PTD-4, TAT-PTD-5, DVP3, DVP6, DVP7, poly-arginine (R9), poly-lysine (K9), FHV coat, signal-peptide I, signal-peptide II, PRES, transportan, amphiphilic model peptide, HSV VP22, and CL22. In some embodiments, the cationic CPP consists of 8-30 amino acids. In one embodiment, the cationic CPP is Tat.

In some embodiments, the therapeutic peptide is an antigenic peptide or a non-immunogenic peptide containing a T-cell epitope. In some embodiments, the T-cell epitope is a tumor-specific epitope or a pathogen-specific epitope. In some embodiments, the therapeutic peptide consists of 9-25 amino acids. The antigenic peptide or the non-immunogenic peptide containing a T-cell epitope is directed to a specific disease, such as a tumor, and an infectious disease.

The nanoparticle of the present invention may further comprise at least one negatively charged molecule non-covalently bound to the CPPs, preferably a negatively charged TLR ligand. In some embodiments, the negatively charged molecule is CpG oligodeoxynucleotides, Poly (I:C), or a combination thereof. In some embodiments, the CpG oligodeoxynucleotides are 20-24 bp in length. In some embodiments, Poly (I:C) is 0.2 to 1 kb in length.

The nanoparticle disclosed herein may also carry at least one hydrophobic molecule non-covalently bound to the therapeutic peptides, preferably a hydrophobic TLR ligand. In some embodiments, the hydrophobic molecule is monophosphoryl lipid A (MPLA), R848, or a combination thereof.

The nanoparticle disclosed herein can be taken up by cells. In some embodiments, the nanoparticle containing antigenic peptides or non-immunogenic peptides with T-cell epitopes can be taken up by APCs—especially DCs or macrophages—both in vitro and in vivo. In some embodiments, the TLR ligand activates one or more TLR signaling pathways.

In another aspect, the present disclosure provides a pharmaceutical composition comprising the nanoparticle of the present invention and a pharmaceutically acceptable carrier of preferably about pH 7.0.

In a third aspect, the present disclosure also provides a composition for generation of the nanoparticles above, comprising cationic CPPs each covalently linked to a hydrophobic therapeutic peptide, and optionally at least one negatively charged molecule and/or at least one hydrophobic molecule.

The components in the composition may be mixed in a medium of about pH 7.0 prior to nanoparticle administration. Nanoparticles are self-assembled in the medium, taken up by APCs in vitro via endocytosis, and then used for vaccines. Alternatively, self-assembled nanoparticles can be prepared, and delivered into animals, where self-assembled nanoparticles are taken up by DCs or macrophages in vivo. Regardless of uptake of nanoparticles by DCs or macrophages in vitro or in vivo, self-assembled nanoparticles will enter endosomes or lysosomes, where nanoparticles will be disrupted at pH 4.5; CPP-linked peptides and TLR ligands are released. While the endosome-localized TLR3, TLR7, TLR8 and TLR9 bind to TLR ligands for triggering innate immune signaling, the CPP-linked peptides will bind to MHC class II molecules in the endosome for loading and presentation, or cross the endosome membrane into the cytoplasmic, ER and Golgi for antigen processing and presentation by MHC class I molecules to T cells.

In some embodiments, the cationic CPP is selected from the group consisting of TAT, TAT-PTD-4, TAT-PTD-5, DVP3, DVP6, DVP7, poly-arginine (R9), poly-lysine (K9), FHV coat, signal-peptide I, signal-peptide II, PRES, transportan, amphiphilic model peptide, HSV VP22, and CL22. In some embodiments, the cationic CPP consists of 8-30 amino acids. In one embodiment, the cationic CPP is Tat. In some embodiments, the therapeutic peptide is an antigenic peptide or a non-immunogenic peptide containing a T-cell epitope. In some embodiments, the T-cell epitope is a tumor-specific epitope or a pathogen-specific epitope. In some embodiments, the therapeutic peptide consists of 8, 9, 10 or 11 amino acids presented by MHC class I molecules, or 9-25 amino acids presented by MHC class II molecules. The antigenic peptide or the non-immunogenic peptide containing a T-cell epitope is directed to a specific disease, such as a tumor, and an infectious disease.

In some embodiments, the negatively charged molecule is a negatively charged TLR ligand. In some embodiments, the negatively charged molecule is CpG oligodeoxynucleotides, Poly(I:C), or the combination thereof. In some embodiments, the CpG oligodeoxynucleotides are 15 to 24 bp in length. In some embodiments, Poly(I:C) is 0.2 to 1 kb in length. In some embodiments, the hydrophobic molecule non-covalently is a hydrophobic TLR ligand. In some embodiments, the hydrophobic molecule is monophosphoryl lipid A (MPLA), R848, or the combination thereof.

In a fourth aspect, the present disclosure provides a method for treatment, prophylaxis, and/or the amelioration of at least one symptom of cancer or an infectious disease. In an overall and general sense, the method typically comprises providing to a subject in need thereof, a therapeutically-effective amount of a pharmaceutical formulation that comprises the nanoparticle disclosed herein.

In some embodiments, the cancer may be a dangerous tumor, and such tumors may be either solid, or non-solid in composition, depending upon the particular disease. In certain embodiments, the cancerous tumor to be treated is a primary tumor or a metastatic tumor, such as, without limitation, one or more melanomas or lung cancers.

In other embodiments, treatment of a disease may be contemplated, particularly, for example, in the treatment of one or more viral, fungal, and/or bacterial infections.

The antigenic peptide or the non-immunogenic peptide containing a T-cell epitope in the nanoparticle is processed and presented by APCs especially DCs or macrophages to T cells by newly synthesized MEW class II molecules for a long period of time. The TLR ligand contained in the nanoparticle stimulates DCs or immune cells to produce innate immune responses such as release of type I interferon cytokines to enhance of capacity of DCs to present an epitope to T cells and to co-stimulate T cells for activation, as well as cytokine stimulation for T cell growth and expansion.

The nanoparticle disclosed herein enables the enhanced co-delivery of antigenic peptides or non-immunogenic peptides containing T-cell epitopes with two or more TLR ligands into the same antigen-presenting cells such as DCs or macrophages, generating potent and effective immune response. The resulting effect, such as an anti-tumor effect or anti-pathogen effect, may be superior to some other delivery platforms, e.g., MSV.

Other objects, features, and advantages of the present disclosure will become apparent from the following detailed descriptions. It should be understood, however, that the detailed descriptions and the specific examples, while indicating preferred embodiments of the invention, are given by way of illustration only, since various changes and modifications within the spirit and scope of the invention will become apparent to those skilled in the art from this detailed description.

### Pharmaceutical Formulations

In certain embodiments, the present disclosure concerns self-assembling nanoparticle compositions prepared in pharmaceutically-acceptable formulations for administration to one or more cells or tissues of an animal, either alone, or in combination with one or more other modalities of diagnosis, prophylaxis and/or therapy. The formulation of pharmaceutically acceptable excipients and carrier solutions is well known to those of ordinary skill in the art, as is the development of suitable dosing and treatment regimens for using the self-assembling nanoparticle compositions described herein in a variety of therapeutic, prophylactic, diagnostic, and prognostic regimens.

In certain circumstances it will be desirable to deliver the disclosed self-assembling nanoparticle compositions in suitably-formulated pharmaceutical vehicles by one or more standard delivery devices, including, without limitation, subcutaneously, parenterally, intravenously, intramuscularly, intrathecally, intratumorally, intraperitoneally, transdermally, topically, by oral or nasal inhalation, or by direct injection to one or more cells, tissues, or organs within or about the body of an animal.

The methods of administration may also include those modalities as described in U.S. Pat. Nos. 5,543,158; 5,641,515, and 5,399,363, each of which is specifically incorporated herein in its entirety by express reference thereto. Solutions of the active compounds as freebase or pharmacologically acceptable salts may be prepared in sterile water, and may be suitably mixed with one or more surfactants, such as hydroxypropylcellulose. Dispersions may also be prepared in glycerol, liquid polyethylene glycols, oils, or mixtures thereof. Under ordinary conditions of storage and use, these preparations contain a preservative to prevent the growth of microorganisms.

For administration of an injectable aqueous solution, without limitation, the solution may be suitably buffered, if necessary, and the liquid diluent first rendered isotonic with sufficient saline or glucose. These particular aqueous solutions are especially suitable for intravenous, intramuscular, subcutaneous, transdermal, subdermal, and/or intraperitoneal administration. In this regard, the compositions of the present invention may be formulated in one or more pharmaceutically acceptable vehicles, including for example sterile aqueous media, buffers, diluents, etc. For example, a given dosage of active ingredient(s) may be dissolved in a particular volume of an isotonic solution (e.g., an isotonic NaCl-based solution), and then injected at the proposed site of administration, or further diluted in a vehicle suitable for intravenous infusion (see, e.g., “REMINGTON'S PHARMACEUTICAL SCIENCES” 15th Edition, pp. 1035-1038 and 1570-1580). While some variation in dosage will necessarily occur depending on the condition of the subject being treated, the extent of the treatment, and the site of administration, the person responsible for administration will nevertheless be able to determine the correct dosing regimens appropriate for the individual subject using ordinary knowledge in the medical and pharmaceutical arts.

Sterile injectable compositions may be prepared by incorporating the disclosed self-assembling nanoparticle compositions in the required amount in the appropriate solvent with several of the other ingredients enumerated above, as required, followed by filtered sterilization. Generally, dispersions can be prepared by incorporating the selected sterilized active ingredient(s) into a sterile vehicle that contains the basic dispersion medium and the required other ingredients from those enumerated above. The self-assembling nanoparticle compositions disclosed herein may also be formulated in a neutral or salt form.

Pharmaceutically acceptable salts include the acid addition salts (formed with the free amino groups of the protein), and which are formed with inorganic acids such as, without limitation, hydrochloric or phosphoric acids, or organic acids such as, without limitation, acetic, oxalic, tartaric, mandelic, and the like. Salts formed with the free carboxyl groups can also be derived from inorganic bases such as, without limitation, sodium, potassium, ammonium, calcium, or ferric hydroxides, and such organic bases as isopropylamine, trimethylamine, histidine, procaine, and the like. Upon formulation, solutions will be administered in a manner compatible with the dosage formulation, and in such amount as is effective for the intended application. Formulations of compounds of the present invention may be administered in a variety of dosage forms such as injectable solutions, topical preparations, oral formulations, including sustain-release capsules, hydrogels, colloids, viscous gels, transdermal reagents, intranasal and inhalation formulations, and the like.

The amount, dosage regimen, formulation, and administration of the self-assembling nanoparticle compositions disclosed herein will be within the purview of the ordinary-skilled artisan having benefit of the present teaching. It is likely, however, that the administration of a diagnostically-effective (i.e., a pharmaceutically-effective) amount of one or more of the disclosed compositions may be achieved by a single administration, such as, without limitation, a single injection of a sufficient quantity of the delivered agent to provide the desired benefit to the patient in need thereof. Alternatively, in some circumstances, it may be desirable to provide multiple, or successive administrations of the disclosed self-assembling nanoparticle compositions, either over a relatively short, or even a relatively prolonged period, as may be determined by the medical practitioner overseeing the administration of such compositions to the selected individual undergoing such procedure(s), treatment, therapy, or diagnosis.

Typically, formulations of one or more of the self-assembling nanoparticle compositions described herein will contain at least an effective amount of a first active agent. Preferably, the formulation may contain at least about 0.001% of each active ingredient, preferably at least about 0.01% of the active ingredient, although the percentage of the active ingredient(s) may, of course, be varied, and may conveniently be present in amounts from about 0.01 to about 90 weight % or volume %, or from about 0.1 to about 80 weight % or volume %, or more preferably, from about 0.2 to about 60 weight % or volume %, based upon the total formulation. Naturally, the amount of active compound(s) in each composition may be prepared in such a way that a suitable dosage will be obtained in any given unit dose of the compound. Factors such as solubility, bioavailability, biological ti/2, route of administration, product shelf life, as well as other pharmacological considerations will be contemplated by one of ordinary skill in the art of preparing such pharmaceutical formulations, and as such, a variety of dosages and treatment regimens may be desirable.

While systemic administration is contemplated to be effective in many embodiments of the invention, it is also contemplated that formulations disclosed herein be suitable for direct injection into one or more organs, tissues, or cell types in the body. Direct administration of the disclosed nanoparticle to particular discreet locations within the body, or directly to tumors, tumor stem cells, cancerous tissues, and/or cancer stem cells, for example, may be conducted using suitable means as known to those of ordinary skill in the relevant medical oncology arts.

Pharmaceutical formulations comprising one or more of the self-assembling nanoparticle compositions disclosed herein may further comprise one or more excipients, buffers, or diluents that are particularly formulated for contact with mammalian cells, and in particular human cells, and/or for administration to a mammalian subject, such as a human patient. Compositions may further optionally comprise one or more diagnostic or prognostic agents, and/or may be formulated with additional population(s) of microspheres, microparticles, nanospheres, or nanoparticles, or may be formulated to contain one or more additional therapeutic and/or diagnostic agent(s), useful in administration to one or more cells, tissues, organs, or body of a mammalian patient (and to a human patient, in particular).

Formulation of pharmaceutically-acceptable excipients and carrier solutions is well-known to those of skill in the art, as is the development of suitable dosing, diagnostic, and/or treatment regimens for using the particular self-assembling nanoparticle compositions described herein in a variety of modalities, including e.g., without limitation, oral, parenteral, intravenous, intranasal, intratumoral, and intramuscular routes of administration.

The particular amount of self-assembling nanoparticle compositions employed, and the particular time of administration, or dosage regimen for compositions employing the disclosed formulations will be within the purview of a person of ordinary skill in the art having benefit of the present teaching. It is likely, however, that the administration of the disclosed formulations may be achieved by administration of one or more doses of the formulation, during a time effective to provide the desired benefit to the patient undergoing such treatment. Such dosing regimens may be determined by the medical practitioner overseeing the administration of the compounds, depending upon the particular condition or the patient, the extent or duration of the therapy being administered, etc.

Pharmaceutical formulations comprising one or more self-assembling nanoparticle compositions as disclosed herein are not in any way limited to use only in humans, or even to primates, or mammals. In certain embodiments, the methods and compositions disclosed herein may be employed using avian, amphibian, reptilian, or other animal species. In preferred embodiments, however, the compositions of the present disclosure are preferably formulated for administration to a mammal, and in particular, to humans, in a variety of regimens for diagnosing, ameliorating, and/or treating one or more diseases within the body of the patient, and particularly, for the treatment of one or more types of tumor or cancer cells, or for treating one or more infections. As noted above, such compositions are not limited only to use in humans, but may also be formulated for veterinary administration, including, without limitation, to selected livestock, exotic or domesticated animals, companion animals (including pets and such like), non-human primates, as well as zoological or otherwise captive specimens, and such like.

Compositions for the Preparation of Medicaments

Another important aspect of the present invention concerns methods for using the disclosed self-assembling nanoparticle compositions (as well as formulations including them) in the preparation of medicaments for preventing, diagnosing, treating and/or ameliorating one or more symptoms of one or more diseases, dysfunctions, abnormal conditions, or disorders in an animal, including, for example, vertebrate mammals. Use of the disclosed self-assembling nanoparticle compositions is particularly contemplated in the diagnosis and/or prognosis of cancer, in the detection and/or prediction of cancer metastasis, or for monitoring the extent thereof, and/or for treatment of one or more abnormal conditions, such as the treatment of one or more cancer cell types in vivo, ex vivo, and/or in situ.

Such use generally involves administration to the mammal in need thereof one or more of the disclosed self-assembling nanoparticle compositions that comprises at least a first active agent, in an amount and for a time sufficient to diagnose, treat, lessen, or ameliorate one or more symptoms of tumor formation, or cancer growth and/or metastasis in an affected mammal. Pharmaceutical formulations including one or more of the disclosed self-assembling nanoparticle compositions also form part of the present disclosure, and particularly those compositions that further include at least a first pharmaceutically-acceptable excipient for use in the therapy and/or amelioration of one or more symptoms of cancer in an affected mammal.

Self-Assembled Nanoparticles

The present disclosure describes the use of cationic cell penetrating peptides (each covalently linked to a hydrophobic therapeutic peptide, and optionally at least one negatively charged molecule and/or at least one hydrophobic molecule) to form a nanoparticle. The resulting nanoparticle has: (i) a core comprising the hydrophobic therapeutic peptides and optionally the hydrophobic molecules, and (ii) a corona comprising the CPPs and optionally the negatively-charged molecules.

Negatively-charged molecules may be a TLR ligand, such as CpG oligodeoxynucleotides, Poly (I:C), DNA, and RNA (mRNA or siRNA). The hydrophobic molecule may preferably be a hydrophobic TLR ligand, which may be monophosphoryl lipid A (MPLA), or R848.

While not wishing to be bound by any theory, it is believed that the self-assembly occurs in an aqueous solution of about pH 7.0 due to hydrophobicity of the therapeutic peptide (and optionally the hydrophobic molecule) and hydrophilicity of CPPs, electric bonds through positively and negatively charged molecule.

The amounts of the components used to form the nanoparticle of the present invention can be determined by those skilled in the art. With more negatively charged molecules, more CPPs will participate in nanoparticle formation. And the amount change of some components may alter the size and shape, as well as in vivo biodistribution of the nanoparticle due to different zeta potentials.

In one embodiment, cationic CPPs (positively charged)-antigenic peptides or weakly immunogenic peptides containing T-cell epitopes (generally hydrophobic) have been designed and synthesized and mixed with CpG oligonucleotides (negatively-charged) and monophosphoryl lipid A (MPLA, hydrophobic) in phosphate buffered saline (PBS).

While both CPP-antigen peptide (10 mM) and CpG (10 mM) were soluble in PBS, but once mixed (1:1), precipitations or aggregates were observed. These aggregates were round nanoparticles, with a diameter of 100 nM in size. The self-assembly was believed to occur through electric interaction of positively charged CPPs with negatively charged molecules, as well as hydrophobic interaction between the peptides themselves and monophosphoryl lipid A (MPLA). Further studies using different ratios (positive:negative charges or molar concentrations) found that different ratios of CPP-therapeutic peptide, CpG and MPLA could produce nanoparticles of 100 nM in size at pH 7, but with different zeta potential (surface charges) and assemble efficiency of each component.

As described above, the nanoparticle of the present invention is self-assembled under neutral condition, e.g., at pH 7.0, and disrupted under acidic condition, e.g., at pH 4.5. Therefore, the nanoparticles are delivered to dendritic cells or macrophages as tight and small-sized particles, and then disrupted inside endosomes where pH becomes 4.5, releasing CPPs with the therapeutic peptides, preferably antigenic peptides or non-immunogenic peptides containing T-cell epitopes, and the other molecules, preferably TLR ligands, into the cytoplasm. Thereafter, the antigenic peptide or the non-immunogenic peptides containing a T-cell epitope are bound to MHC class I or II molecules and the epitopes are presented to T-cells, while the TLR ligands bind to TLRs to trigger TLR-mediated signaling pathways (NF-κB, and type I interferon), producing pro-inflammatory cytokines and inducing strong innate and adaptive immune responses.

### Chemotherapeutic Methods and Use

An important aspect of the present disclosure concerns methods for using the disclosed self-assembling nanoparticle formulations for treating or ameliorating the symptoms of one or more forms of cancer, including, for example, a tumor or a metastatic cancer, such as, without limitation, melanoma metastasis to the mammalian lung. Such methods generally involve administering to a mammal (and in particular, to a human in need thereof), one or more of the disclosed self-assembling nanoparticle compositions comprising at least a first anticancer therapeutic, in an amount and for a time sufficient to treat (or, alternatively ameliorate one or more symptoms of) the cancer in an affected mammal.

In certain embodiments, the self-assembling nanoparticle compositions described herein may be provided to the animal in a single treatment modality (either as a single administration, or alternatively, in multiple administrations over a period of from several hours (hrs) to several days (or even several weeks or several months) as needed to treat the particular disease, disorder, dysfunction, or abnormal condition. Alternatively, in some embodiments, it may be desirable to continue the treatment, or to include it in combination with one or more additional modes of therapy, for a period of several months or longer. In other embodiments, it may be desirable to provide the therapy in combination with one or more conventional treatment regimens.

The present disclosure also provides for the use of one or more of the disclosed self-assembling nanoparticle compositions in the manufacture of a medicament for therapy and/or for the amelioration of one or more symptoms of infection or cancer, and particularly for use in the manufacture of a medicament for treating and/or ameliorating one or more symptoms of a mammalian infection or cancer, including, for example human infections, cancerous tumors, and the linker.

The present invention also provides for the use of one or more of the disclosed self-assembling nanoparticle compositions in the manufacture of a medicament for the treatment of a disease or disorder in a mammal, and in particular, for the treatment of one or more human diseases such as an infection and/or cellular hyperproliferation (i.e., cancer).

### Therapeutic Kits

Therapeutic kits including one or more of the disclosed self-assembling nanoparticle compositions and instructions for using the kit in a particular treatment modality also represent preferred aspects of the present disclosure. These kits may further optionally include one or more additional therapeutic compounds, one or more diagnostic reagents, or any combination thereof.

The kits of the invention may be packaged for commercial distribution, and may further optionally include one or more delivery devices adapted to deliver self-assembling nanoparticle composition(s) to an animal (e.g., syringes, injectables, and the like). Such kits typically include at least one vial, test tube, flask, bottle, syringe, or other container, into which the self-assembling nanoparticle composition(s) may be placed, and preferably suitably aliquotted. Where a second pharmaceutical is also provided, the kit may also contain a second distinct container into which this second composition may be placed. Alternatively, a plurality of self-assembling nanoparticles as disclosed herein may be prepared in a single mixture, such as a suspension or solution, and may be packaged in a single container, such as a vial, flask, syringe, catheter, cannula, bottle, or other suitable single container.

The kits of the present invention may also typically include a retention mechanism adapted to contain or retain the vial(s) or other container(s) in close confinement for commercial sale, such as, e.g., injection or blow-molded plastic containers into which the desired vial(s) or other container(s) may be retained to minimize or prevent breakage, exposure to sunlight, or other undesirable factors, or to permit ready use of the composition(s) included within the kit.

Cell-Penetrating Peptides

Cell-penetrating amphiphilic peptides such as HIV-Tat based peptides and chimeric cell-penetrating peptides have also been applied for the delivery of therapeutic cargos to their targets (Magzoub et al., 2004).

CPPs have long been used as drug delivery vehicles because they are able to translocate the cell membrane (Gupta et al., 2005). CPPs, which are cationic short peptides of fewer than 30 amino acids, and poly-arginine-based CPPs the length of 8-10 arginine residues have shown the most efficient membrane penetration (Fuchs et al., 2006).

From the classic mechanism (Fuchs et al., 2006) the membrane penetration of CPPs is based on the hydrogen bonding interaction between the guanidinium groups of arginine residues and the carboxyl, phosphoryl, or sulfuryl groups of the carbohydrates and phospholipids on the cell surface. Initially the pathway of CPP translocation through membranes was defined via receptor- and endocytosis-independent mechanisms, but now, a novel CPP internalization mechanism has been demonstrated.

Several CPPs have been identified, from proteins, including the Tat protein of human immunodeficiency virus (HIV), the VP22 protein of herpes simplex virus, and the fibroblast growth factor.

Some examples of cell penetrating peptides include, but are not limited to:

| | 1. (HIV Tat 47-57) |
|---|---|
| (SEQ ID NO: 3) | |
| | YGRKKRRQRRR; |
| | 2 (TAT-PTD-4) |
| (SEQ ID NO: 4) | |
| | YARAAARQARA; |
| | 3. (TAT-PTD-5) |
| (SEQ ID NO: 5) | |
| | YARAARRAARR; |
| | 4. (DPV3) |
| (SEQ ID NO: 6) | |
| | RKKRRRESRKKRRRES; |
| | 5. (DPV6) |
| (SEQ ID NO: 7) | |
| | GRPRESGKKRKRKRLKP; |
| | 6. (DPV7) |
| (SEQ ID NO: 8) | |
| | GKRKKKGKLGKKRDP; |
| | 7. (poly-Arginine, R9) |
| (SEQ ID NO: 9) | |
| | RRRRRRRRR; |
| | 8. (poly-Lysine, K9) |
| (SEQ ID NO: 10) | |
| | KKKKKKKKK; |
| | 8. (FHV coat) |
| (SEQ ID NO: 11) | |
| | RRRRNRTRRNRRRVR; |
| | 9. (Signal-peptide II) |
| (SEQ ID NO: 12) | |
| | GALFLGWLGAAGSTMGAWSQPKKKRKV; |
| | 10. (Amphiphilic model peptide) |
| (SEQ ID NO: 13) | |
| | KLALKLALKALKAALKLA; |
| | 11. (HSV VP22) |
| (SEQ ID NO: 14) | |
| | DAATATRGRSAASRPTERPRAPARSASRPRRPVE; |
| | 12. (peptide carrier) |
| (SEQ ID NO: 15) | |
| | KETWWETWWTEWSQPKKKRKV; |
| | and |
| | 13. (CL22) |
| (SEQ ID NO: 16) | |
| | KKKKKKGGFLGFWRGENGRKTRSAYERMCNILKGK. |

A comprehensive list of known CPPs can be found online in the publicly-available CPPsite 2.0 website, which is an updated version of the database (CPPsite) of cell-penetrating peptides.

In the present disclosure, cationic CPPs are covalently linked to a T cell epitope peptide, and present at the corona part of the nanoparticle. The positively charged or near neutral corona makes the nanoparticle attach to the negatively charged cell surface and then taken up by cells such as DCs or macrophages with improved uptake efficiency.

The CPPs also help the antigenic peptide or the weak-immunogenic peptide containing a T-cell epitope linked thereto to directly bind to MHC class II molecules in the endosomes for antigen presentation on the cell surface or to escape from the endosome and then enter ER and Golgi where the antigenic peptide or the non-immunogenic peptide containing a T-cell epitope binds to newly synthesized MHC class I molecules for presentation on the cell surface for T cell activation.

A cationic CPP is required in the present invention, as described above. However, a CPP that is not cationic may be modified by adding or attaching some amino acids such Lys, Arg and His to the backbone chain, as well known by those skilled in the art. For example, Poly-Lys or Arg peptides are synthesized as cationic CPPs.

Antigenic Peptides

In the context of the present disclosure, the term “antigenic peptide” refers to a peptide antigen that is common to a specific tumor or a pathogen and binds to MHC molecules.

The tumor antigens of the present disclosure are preferably derived from cancers including, but not limited to, primary or metastatic melanoma, thymoma, lymphoma, sarcoma, lung cancer, liver cancer, non-Hodgkin's lymphoma, Hodgkin's lymphoma, leukemias, uterine cancer, cervical cancer, bladder cancer, kidney cancer and adenocarcinomas, such as breast cancer, prostate cancer, ovarian cancer, pancreatic cancer, and the like. In one embodiment, the tumor antigens of the present disclosure comprise one or more antigenic cancer epitopes immunologically recognized by tumor infiltrating lymphocytes (TIL) derived from a cancerous tumor of a mammal.

Malignant tumors express a number of peptides that can serve as target antigens for an immune attack. These molecules include, but are not limited to, tissue-specific antigens, such as MART-1, tyrosinase and GP 100 in melanoma and prostatic acid phosphatase (PAP) and prostate-specific antigen (PSA) in prostate cancer⁴. Other target molecules belong to the group of transformation-related molecules such as the oncogene HER-2/Neu/ErbB-2. Yet another group of target antigens are onco-fetal antigens such as carcino-embryonic antigen (CEA).

Examples of tumor antigens such as cancer-testis antigens and mutation-derived neoantigens, include but are not limited to NY-ESO-1, CT83, MAGE gene family and neoantigens⁴.

Likewise, oncogene product peptide antigens have been identified that are common to specific tumor types. These polypeptides will find use in the polypeptide complexes of the present invention as reagents that can be used generally to stimulate T-cell responses effective to react with tumors bearing such antigens, oncogene product peptide antigens include, but are not limited to, HER-2/neu associated with human breast and gynecological cancers, carcinoembryonic antigen (CEA) associated with cancer of the pancreas.

The tumor antigen and the antigenic cancer epitopes thereof may be purified and isolated from natural sources such as from primary clinical isolates, cell lines and the like. The cancer peptides and their antigenic epitopes may also be obtained by chemical synthesis or by recombinant DNA techniques known in the arts. Techniques for chemical synthesis are described in Steward et al. (1969); Bodansky et al. (1976); Meienhofer (1983); and Schroder et al. (1965).

Furthermore, as described in Renkvist et al. (2001), there are numerous antigens known in the art. A variety of T cell-defined epitopes encoded by tumor antigens and recognized by T cells (either cytotoxic CD8⁺ or helper CD4⁺) are listed in PCT Intl. Pat. Appl. Publ. No. WO 02/064057, which is specifically incorporated herein in its entirety by express reference thereto.

Although analogs or artificially modified epitopes are not listed, a skilled artisan recognizes how to obtain or generate them by standard means in the art. Other antigens, identified by antibodies and as detected by the Serex technology [see Sahin et al. (1997) and Chen et al. (2000)], are identified in the database of the Ludwig Institute for Cancer Research, which a skilled artisan can easily find on the World Wide Web.

The antigenic peptide of the present invention is required to be hydrophobic so that it is enclosed in the nanoparticle and delivered to the endosome. An antigenic peptide that is not hydrophobic may be modified by adding or attaching one or more amino acids such as Gly, Ala, Val, Leu, Ile, Pro, Phe, Met and Trp to the backbone chain, as well known by those skilled in the art, to increase the antigenicity of the peptide.

T-Cell Epitopes

The immunogenic peptide forming the core part of the nanoparticle of the present invention comprises a T-cell epitope.

Since the T-cell epitopes do not need to be displayed on the surface of a carrier to cause immunization, they can be incorporated into the core of the nanoparticle.

The T-cell epitopes can be chosen from different sources. For example, the T-cell epitopes can be determined by experimental methods. Such epitopes are known in the literature, and can also be predicted by algorithms based on existing protein sequences of a particular pathogen or a cancer antigen, or they may be designed de novo.

There is a wealth of known T-cell epitopes available in the scientific literature. These T-cell epitopes can be selected from a particular pathogen, from a cancer specific peptide sequence, or they may be de novo designed peptides with a particular feature, e.g., the PADRE peptide (see e.g., U.S. Pat. No. 5,736,142, which is specifically incorporated herein in its entirety by express reference thereto) that binds to many different MHC II molecules, which makes it a so-called promiscuous T-cell epitope. There exist commonly accessible databases that contain thousands of different T-cell epitopes, for example the MHC-database “MHCBN VERSION 4.0” or the PDB-database “Protein Data Bank,” or others.

It is well known and well documented that incorporation of helper T cells (HTLs) epitopes into an otherwise not immunogenic peptide sequence or attaching it to a non-peptidic antigen can make those much more immunogenic. The Pan-DR binding peptide HTL epitope PADRE has widely been used in vaccine design for a malaria, Alzheimer's and many other vaccines.

According to the definition of the MHCBN database (supra), T-cell epitopes are peptides that have binding affinities (IC50 values) of less than 50,000 nM to the corresponding MHC molecule. Such peptides are considered as MEW binders. According to this definition, as of August 2006, in the Version 4.0 of the MHCBN database the following data is available: 20717 MEW binders and 4022 MHC non-binders.

Suitable T-cell epitopes can also be obtained by using prediction algorithms. These prediction algorithms can either scan an existing protein sequence from a pathogen or a cancer antigen for putative T-cell epitopes, or they can predict, whether de novo designed peptides bind to a particular MHC molecule. Many such prediction algorithms are commonly accessible on the internet. Examples are SVRMHCdb (Wan et al., 2006), SYFPEITHI, MHCPred, motif scanner or NetMHCIIpan for MEW II binding molecules and NetMHCpan for MEW I binding epitopes.

HTL epitopes as described herein and preferred for the design are peptide sequences that are either measured by biophysical methods or predicted by NetMHCIIpan to bind to any of the MHC II molecules with binding affinities (IC50 values) better than 500 nM. These are considered weak binders. Preferentially these epitopes are measured by biophysical methods or predicted by NetMHCIIpan to bind to the MHC II molecules with IC50 values better than 50 nM. These are considered strong binders.

The T-cell epitopes can be incorporated at several places within the non-immunogenic peptide. To achieve this, the particular sequence with the T-cell epitope has to obey the rules for MHC binding. The rules for binding to MEW molecules are incorporated into the MHC binding prediction programs that use sophisticated algorithms to predict MHC binding peptides.

There are many different HLA molecules, each of them having a restriction of amino acids in their sequence that will best bind to it. The binding motifs are summarized in Table 3 of U.S. Pat. No. 8,546,337, which is specifically incorporated herein in its entirety by express reference thereto. In that table the motif shows x for positions that can have any amino acid, and in square brackets the (list of) amino acids that can only be at a particular position of the binding motif.

Generally, in the present invention, the non-immunogenic peptide containing a T-cell epitope is required to be hydrophobic so that it is enclosed in the nanoparticle and delivered to the endosome. A non-immunogenic peptide containing a T-cell epitope this is not hydrophobic may be modified by adding or attaching some amino acids such as Gly, Ala, Val, Leu, Ile, Pro, Phe, Met and Trp to the backbone chain, as well known by those skilled in the art.

Toll-Like Receptor and TLR Signaling

Toll-like Receptors (TLRs) are evolutionarily conserved receptors and are homologues of the Drosophila Toll protein, which was found to be important for defense against microbial infection. TLRs recognize highly conserved structural motifs known as pathogen-associated microbial patterns (PAMPs), which are exclusively expressed by microbial pathogens, or danger-associated molecular patterns (DAMPs) that are endogenous molecules released from necrotic or dying cells.

The TLRs include TLR1, TLR2, TLR3, TLR4, TLRS, TLR6, TLR7, TLR8, TLR9, TLR10, TLR11, TLR12, and TLR13, though the latter two are not found in humans.

TLRs are expressed in innate immune cells such as dendritic cells (DCs) and macrophages as well as non-immune cells such as fibroblast cells and epithelial cells. TLRs are largely classified into two subfamilies based on their localization, cell surface TLRs and intracellular TLRs. Cell surface TLRs include TLR1, TLR2, TLR4, TLR5, TLR6, and TLR10, whereas intracellular TLRs are localized in the endosome and include TLR3, TLR7, TLR8, TLR9, TLR11, TLR12, and TLR13.

Stimulation of TLRs by the corresponding PAMPs or DAMPs initiates signaling cascades leading to the activation of transcription factors, such as AP-1, NF-κB and interferon regulatory factors (IRFs). Signaling by TLRs result in a variety of cellular responses including the production of interferons (IFNs), pro-inflammatory cytokines and effector cytokines that direct the adaptive immune response.

TLR signaling consists of at least two distinct pathways: a MyD88-dependent pathway that leads to the production of inflammatory cytokines, and a TRIF-dependent pathway associated with the stimulation of IFN-β and the maturation of dendritic cells.

TLR Ligand

Because of the specificity of TLRs (and other innate immune receptors), they cannot be easily changed over the course of evolution; these receptors recognize molecules that are constantly associated with threats (e.g., pathogens or cell stress), and are highly specific to these threats.

Pathogen-associated molecules that meet this requirement are thought to be critical to the pathogen's function and difficult to change through mutation; they are said to be evolutionarily conserved. Somewhat conserved features in pathogens include bacterial cell-surface lipopolysaccharides (LPS), lipoproteins, lipopeptides, and lipoarabinomannan; proteins such as flagellin from bacterial flagella; double-stranded RNA of viruses; or the unmethylated CpG islands of bacterial and viral DNA; and also of the CpG islands found in the promoters of eukaryotic DNA; as well as certain other RNA and DNA molecules.

CpG-A and CpG-B

Table 1 lists some well-known TLRs and their common ligands:

TABLE 1
Commonly-Known TLRs and Their Ligands

| | | | Receptor | |
|---|---|---|---|---|
| Receptor | Ligand(s) | Ligand location | location | Cell types |
| TLR 1 | multiple triacyl | Bacterial | cell surface | monocytes/macrophages |
| | lipopeptides | lipoprotein | | a subset of dendritic cells |
| | | | | B lymphocytes |
| TLR 2 | multiple glycolipids | Bacterial | cell surface | monocytes/macrophages |
| | | peptidoglycans | | neutrophils |
| | multiple lipopeptides | Bacterial | | Myeloid dendritic cells |
| | | peptidoglycans | | Mast cells |
| | multiple lipoproteins | Bacterial | | |
| | | peptidoglycans | | |
| | lipoteichoic acid | Gram-positive | | |
| | | bacteria | | |
| | HSP70 | Host cells | | |
| | zymosan (Beta-glucan) | Fungi | | |
| | Numerous others | | | |
| TLR 3 | double-stranded RNA, | viruses | cell | Dendritic cells |
| | poly I:C | | compartment | B lymphocytes |
| TLR 4 | lipopolysaccharide | Gram-negative | cell surface | monocytes/macrophages |
| | | bacteria | | neutrophils |
| | several heat shock proteins | Bacteria and | | Myeloid dendritic cells |
| | | host cells | | Mast cells |
| | fibrinogen | host cells | | B lymphocytes |
| | heparan sulfate fragments | host cells | | Intestinal epithelium |
| | hyaluronic acid fragments | host cells | | Breast cancer cells |
| | nickel | | | |
| | Various opioid drugs | | | |
| TLR 5 | Bacterial flagellin | Bacteria | cell surface | monocyte/macrophages |
| | Profilin | Toxoplasma | | a subset of dendritic cells |
| | | gondii | | Intestinal epithelium |
| | | | | Breast cancer cells |
| TLR 6 | multiple diacyl | Mycoplasma | cell surface | monocytes/macrophages |
| | lipopeptides | | | Mast cells |
| | | | | B lymphocytes |
| TLR 7 | imidazoquinoline | small synthetic | cell | monocytes/macrophages |
| | loxoribine (a guanosine | compounds | compartment | Plasmacytoid dendritic |
| | analogue) | | | cells |
| | bropirimine | | | B lymphocytes |
| | single-stranded RNA | RNA viruses | | |
| TLR 8 | small synthetic | | cell | monocytes/macrophages |
| | compounds; single- | | compartment | a subset of dendritic cells |
| | stranded Viral RNA, | | | Mast cells |
| | phagocytized bacterial | | | Intestinal epithelial cells |
| | RNA | | | (IECs) *only in Crohn's or |
| | | | | ulcerative colitis |
| TLR 9 | unmethylated CpG | Bacteria, DNA | cell | monocytes/macrophages |
| | Oligodeoxynucleotide | viruses | compartment | Plasmacytoid dendritic |
| | DNA | | | cells |
| | | | | B lymphocytes |
| TLR 10 | Unknown | | | |
| TLR 11 | Profilin | Toxoplasma | cell | monocytes/macrophages |
| | | gondii | compartment | liver cells |
| | | | | kidney |
| | | | | urinary bladder epithelium |
| TLR 12 | Profilin | Toxoplasma | | Neurons |
| | | gondii | | plasmacytoid dendritic |
| | | | | cells |
| | | | | conventional dendritic cells |
| | | | | macrophages |
| TLR 13 | bacterial ribosomal RNA | Virus, bacteria | cell | monocytes/macrophages |
| | sequence | | compartment | conventional dendritic cells |
| | “CGGAAAGACC” | | | |

The nanoparticles of the present invention are to be taken up by antigen-presenting cells (APCs) especially dendritic cells (DCs) or macrophages where TLRs are diversely distributed. Therefore, to trigger robust T cell responses, the ligands in Table 1 and others described herein or elsewhere may be formed the nanoparticles. For different diseases to be treated or prevented, TLRs may be selected for nanoparticle formation depending on the main type of antigen-presenting cells involved in the immune response.

One or more, preferably two or more TLR ligands may be contained in one nanoparticle. The hydrophobic TLR ligands may be located at the core part of the nanoparticle together with the hydrophobic therapeutic peptide, while the negatively charged TLR ligands may be present at the corona part of the nanoparticle with the cationic CPPs. The electric interaction of positively charged CPPs with negatively charged TLR ligands are believed to provide a more stable and tight nanostructure. In one embodiment, two negatively charged TLR ligands are contained at the corona part of the nanoparticle. In another embodiment, one hydrophobic TLR ligand is contained at the core part of the nanoparticle, and two negatively charged TLR ligands are contained at the corona part of the nanoparticle. The presence of multiple types of TLR ligands in the nanoparticle evidently improves the T cell responses. The TLR ligands can also be modified to have desired properties, as well known by those skilled in the art.

### Exemplary Definitions

In accordance with the present invention, polynucleotides, nucleic acid segments, nucleic acid sequences, and the like, include, but are not limited to, DNAs (including and not limited to genomic or extragenomic DNAs), genes, peptide nucleic acids (PNAs) RNAs (including, but not limited to, rRNAs, mRNAs and tRNAs), nucleosides, and suitable nucleic acid segments either obtained from natural sources, chemically synthesized, modified, or otherwise prepared or synthesized in whole or in part by the hand of man.

Unless defined otherwise, all technical and scientific terms used herein have the same meaning as commonly understood by one of ordinary skill in the art to which this invention belongs. The following references provide one of skill with a general definition of many of the terms used in this invention: Dictionary of Biochemistry and Molecular Biology, (2^{nd} Ed.) J. Stenesh (Ed.), Wiley-Interscience (1989); Dictionary of Microbiology and Molecular Biology (3^{rd} Ed.), P. Singleton and D. Sainsbury (Eds.), Wiley-Interscience (2007); Chambers Dictionary of Science and Technology (2^{nd} Ed.), P. Walker (Ed.), Chambers (2007); Glossary of Genetics (5^{th} Ed.), R. Rieger et al. (Eds.), Springer-Verlag (1991); and The HarperCollins Dictionary of Biology, W. G. Hale and J. P. Margham, (Eds.), HarperCollins (1991).

Although any methods and compositions similar or equivalent to those described herein can be used in the practice or testing of the present invention, the preferred methods, and compositions are described herein. For purposes of the present invention, the following terms are defined below for sake of clarity and ease of reference:

In accordance with long standing patent law convention, the words “a” and “an,” when used throughout this application and in the claims, denote “one or more.”

The terms “about” and “approximately” as used herein, are interchangeable, and should generally be understood to refer to a range of numbers around a given number, as well as to all numbers in a recited range of numbers (e.g., “about 5 to 15” means “about 5 to about 15” unless otherwise stated). Moreover, all numerical ranges herein should be understood to include each whole integer within the range.

Biocompatible” refers to a material that, when exposed to living cells, will support an appropriate cellular activity of the cells without causing an undesirable effect in the cells, such as a change in a living cycle of the cells, a change in a proliferation rate of the cells, or a cytotoxic effect.

The term “biologically-functional equivalent” is well understood in the art, and is further defined in detail herein. Accordingly, sequences that have about 85% to about 90%; or more preferably, about 91% to about 95%; or even more preferably, about 96% to about 99%; of nucleotides that are identical or functionally-equivalent to one or more of the nucleotide sequences provided herein are particularly contemplated to be useful in the practice of the methods and compositions set forth in the instant application.

As used herein, “biomimetic” shall mean a resemblance of a synthesized material to a substance that occurs naturally in a human body and which is not rejected by (e.g., does not cause an adverse reaction in) the human body.

As used herein, the term “buffer” includes one or more compositions, or aqueous solutions thereof, that resist fluctuation in the pH when an acid or an alkali is added to the solution or composition that includes the buffer. This resistance to pH change is due to the buffering properties of such solutions, and may be a function of one or more specific compounds included in the composition. Thus, solutions or other compositions exhibiting buffering activity are referred to as buffers or buffer solutions. Buffers generally do not have an unlimited ability to maintain the pH of a solution or composition; rather, they are typically able to maintain the pH within certain ranges, for example from a pH of about 5 to 7.

As used herein, the term “carrier” is intended to include any solvent(s), dispersion medium, coating(s), diluent(s), buffer(s), isotonic agent(s), solution(s), suspension(s), colloid(s), inert(s) or such like, or a combination thereof, that is pharmaceutically acceptable for administration to the relevant animal. The use of one or more delivery vehicles for chemical compounds in general, and chemotherapeutics in particular, is well known to those of ordinary skill in the pharmaceutical arts. Except insofar as any conventional media or agent is incompatible with the active ingredient, its use in the diagnostic, prophylactic, and therapeutic compositions is contemplated. One or more supplementary active ingredient(s) may also be incorporated into, or administered in association with, one or more of the disclosed chemotherapeutic compositions.

As used herein, the term “DNA segment” refers to a DNA molecule that has been isolated free of total genomic DNA of a particular species. Therefore, a DNA segment obtained from a biological sample using one of the compositions disclosed herein refers to one or more DNA segments that have been isolated away from, or purified free from, total genomic DNA of the particular species from which they are obtained. Included within the term “DNA segment,” are DNA segments and smaller fragments of such segments, as well as recombinant vectors, including, for example, plasmids, cosmids, phage, viruses, and the like.

The term “effective amount,” as used herein, refers to an amount that is capable of treating or ameliorating a disease or condition or otherwise capable of producing an intended therapeutic effect.

The term “for example” or “e.g.,” as used herein, is used merely by way of example, without limitation intended, and should not be construed as referring only those items explicitly enumerated in the specification.

As used herein, a “heterologous” sequence is defined in relation to a predetermined, reference sequence, such as, a polynucleotide or a polypeptide sequence. For example, with respect to a structural gene sequence, a heterologous promoter is defined as a promoter which does not naturally occur adjacent to the referenced structural gene, but which is positioned by laboratory manipulation. Likewise, a heterologous gene or nucleic acid segment is defined as a gene or segment that does not naturally occur adjacent to the referenced promoter and/or enhancer elements.

As used herein, “homologous” means, when referring to polynucleotides, sequences that have the same essential nucleotide sequence, despite arising from different origins. Typically, homologous nucleic acid sequences are derived from closely related genes or organisms possessing one or more substantially similar genomic sequences. By contrast, an “analogous” polynucleotide is one that shares the same function with a polynucleotide from a different species or organism, but may have a significantly different primary nucleotide sequence that encodes one or more proteins or polypeptides that accomplish similar functions or possess similar biological activity. Analogous polynucleotides may often be derived from two or more organisms that are not closely related (e.g., either genetically or phylogenetically).

As used herein, the term “homology” refers to a degree of complementarity between two or more polynucleotide or polypeptide sequences. The word “identity” may substitute for the word “homology” when a first nucleic acid or amino acid sequence has the exact same primary sequence as a second nucleic acid or amino acid sequence. Sequence homology and sequence identity can be determined by analyzing two or more sequences using algorithms and computer programs known in the art. Such methods may be used to assess whether a given sequence is identical or homologous to another selected sequence.

The terms “identical” or percent “identity,” in the context of two or more nucleic acid or polypeptide sequences, refer to two or more sequences or subsequences that are the same or have a specified percentage of amino acid residues or nucleotides that are the same, when compared and aligned for maximum correspondence, as measured using one of the sequence comparison algorithms described below (or other algorithms available to persons of ordinary skill) or by visual inspection.

As used herein, the phrase “in need of treatment” refers to a judgment made by a caregiver such as a physician or veterinarian that a patient requires (or will benefit in one or more ways) from treatment. Such judgment may made based on a variety of factors that are in the realm of a caregiver's expertise, and may include the knowledge that the patient is ill as the result of a disease state that is treatable by one or more compound or pharmaceutical compositions such as those set forth herein.

The phrases “isolated” or “biologically pure” refer to material that is substantially, or essentially, free from components that normally accompany the material as it is found in its native state.

As used herein, the term “kit” may be used to describe variations of the portable, self-contained enclosure that includes at least one set of reagents, components, or pharmaceutically-formulated compositions to conduct one or more of the assay methods of the present invention. Optionally, such kit may include one or more sets of instructions for use of the enclosed reagents, such as, for example, in a laboratory or clinical application.

“Link” or “join” refers to any method known in the art for functionally connecting one or more proteins, peptides, nucleic acids, or polynucleotides, including, without limitation, recombinant fusion, covalent bonding, disulfide bonding, ionic bonding, hydrogen bonding, electrostatic bonding, and the like.

The term “naturally-occurring” as used herein as applied to an object refers to the fact that an object can be found in nature. For example, a polypeptide or polynucleotide sequence that is present in an organism (including viruses) that can be isolated from a source in nature and which has not been intentionally modified by the hand of man in a laboratory is naturally-occurring. As used herein, laboratory strains of rodents that may have been selectively bred according to classical genetics are considered naturally-occurring animals.

As used herein, the term “nucleic acid” includes one or more types of: polydeoxyribonucleotides (containing 2-deoxy-D-ribose), polyribonucleotides (containing D-ribose), and any other type of polynucleotide that is an N-glycoside of a purine or pyrimidine base, or modified purine or pyrimidine bases (including abasic sites). The term “nucleic acid,” as used herein, also includes polymers of ribonucleosides or deoxyribonucleosides that are covalently bonded, typically by phosphodiester linkages between subunits, but in some cases by phosphorothioates, methylphosphonates, and the like. “Nucleic acids” include single- and double-stranded DNA, as well as single- and double-stranded RNA. Exemplary nucleic acids include, without limitation, gDNA; hnRNA; mRNA; rRNA, tRNA, micro RNA (miRNA), small interfering RNA (siRNA), small nucleolar RNA (snORNA), small nuclear RNA (snRNA), and small temporal RNA (stRNA), and the like, and any combination thereof.

The terms “operably linked” and operatively linked”, as used herein, refers to that union of the nucleic acid sequences that are linked in such a way, such that the coding regions are contiguous and in correct reading frame. Such sequences are typically contiguous, or substantially contiguous. However, since enhancers generally function when separated from the promoter by several kilobases and intronic sequences may be of variable lengths, some polynucleotide elements may be operably linked but not contiguous.

As used herein, the term “patient” (also interchangeably referred to as “host” or “subject”) refers to any host that can receive one or more of the pharmaceutical compositions disclosed herein. Preferably, the subject is a vertebrate animal, which is intended to denote any animal species (and preferably, a mammalian species such as a human being). In certain embodiments, a “patient” refers to any animal host including without limitation any mammalian host. Preferably, the term refers to any mammalian host, the latter including but not limited to, human and non-human primates, bovines, canines, caprines, cavines, corvines, epines, equines, felines, hircines, lapines, leporines, lupines, murines, ovines, porcines, ranines, racines, vulpines, and the like, including livestock, zoological specimens, exotics, as well as companion animals, pets, and any animal under the care of a veterinary practitioner. A patient can be of any age at which the patient is able to respond to inoculation with the present vaccine by generating an immune response. In particular embodiments, the mammalian patient is preferably human.

The phrase “pharmaceutically-acceptable” refers to molecular entities and compositions that preferably do not produce an allergic or similar untoward reaction when administered to a mammal, and in particular, when administered to a human.

As used herein, “pharmaceutically acceptable salt” refers to a salt that preferably retains the desired biological activity of the parent compound and does not impart any undesired toxicological effects. Examples of such salts include, without limitation, acid addition salts formed with inorganic acids (e.g., hydrochloric acid, hydrobromic acid, sulfuric acid, phosphoric acid, nitric acid, and the like); and salts formed with organic acids including, without limitation, acetic acid, oxalic acid, tartaric acid, succinic acid, maleic acid, fumaric acid, gluconic acid, citric acid, malic acid, ascorbic acid, benzoic acid, tannic acid, pamoic (embonic) acid, alginic acid, naphthoic acid, polyglutamic acid, naphthalenesulfonic acids, naphthalenedisulfonic acids, polygalacturonic acid; salts with polyvalent metal cations such as zinc, calcium, bismuth, barium, magnesium, aluminum, copper, cobalt, nickel, cadmium, and the like; salts formed with an organic cation formed from N,N′-dibenzylethylenediamine or ethylenediamine; and combinations thereof.

As used herein, the term “plasmid” or “vector” refers to a genetic construct that is composed of genetic material (i.e., nucleic acids). Typically, a plasmid or a vector contains an origin of replication that is functional in bacterial host cells, e.g., Escherichia coli, and selectable markers for detecting bacterial host cells including the plasmid. Plasmids and vectors of the present invention may include one or more genetic elements as described herein arranged such that an inserted coding sequence can be transcribed and translated in a suitable expression cells. In addition, the plasmid or vector may include one or more nucleic acid segments, genes, promoters, enhancers, activators, multiple cloning regions, or any combination thereof, including segments that are obtained from or derived from one or more natural and/or artificial sources.

As used herein, “polymer” means a chemical compound or mixture of compounds formed by polymerization and including repeating structural units. Polymers may be constructed in multiple forms and compositions or combinations of compositions.

As used herein, the term “polypeptide” is intended to encompass a singular “polypeptide” as well as plural “polypeptides,” and includes any chain or chains of two or more amino acids. Thus, as used herein, terms including, but not limited to “peptide,” “dipeptide,” “tripeptide,” “protein,” “enzyme,” “amino acid chain,” and “contiguous amino acid sequence” are all encompassed within the definition of a “polypeptide,” and the term “polypeptide” can be used instead of, or interchangeably with, any of these terms. The term further includes polypeptides that have undergone one or more post-translational modification(s), including for example, but not limited to, glycosylation, acetylation, phosphorylation, amidation, derivatization, proteolytic cleavage, post-translation processing, or modification by inclusion of one or more non-naturally occurring amino acids. Conventional nomenclature exists in the art for polynucleotide and polypeptide structures.

For example, one-letter and three-letter abbreviations are widely employed to describe amino acids: Alanine (A; Ala), Arginine (R; Arg), Asparagine (N; Asn), Aspartic Acid (D; Asp), Cysteine (C; Cys), Glutamine (Q; Gln), Glutamic Acid (E; Glu), Glycine (G; Gly), Histidine (H; His), Isoleucine (I; Ile), Leucine (L; Leu), Methionine (M; Met), Phenylalanine (F; Phe), Proline (P; Pro), Serine (S; Ser), Threonine (T; Thr), Tryptophan (W; Trp), Tyrosine (Y; Tyr), Valine (V; Val), and Lysine (K; Lys). Amino acid residues described herein are preferred to be in the “L” isomeric form. However, residues in the “D” isomeric form may be substituted for any L-amino acid residue provided the desired properties of the polypeptide are retained.

As used herein, the terms “prevent,” “preventing,” “prevention,” “suppress,” “suppressing,” and “suppression” as used herein refer to administering a compound either alone or as contained in a pharmaceutical composition prior to the onset of clinical symptoms of a disease state so as to prevent any symptom, aspect or characteristic of the disease state. Such preventing and suppressing need not be absolute to be deemed medically useful.

“Protein” is used herein interchangeably with “peptide” and “polypeptide,” and includes both peptides and polypeptides produced synthetically, recombinantly, or in vitro and peptides and polypeptides expressed in vivo after nucleic acid sequences are administered into a host animal or human subject. The term “polypeptide” is preferably intended to refer to any amino acid chain length, including those of short peptides from about 2 to about 20 amino acid residues in length, oligopeptides from about 10 to about 100 amino acid residues in length, and longer polypeptides including from about 100 amino acid residues or more in length. Furthermore, the term is also intended to include enzymes, i.e., functional biomolecules including at least one amino acid polymer. Polypeptides and proteins of the present invention also include polypeptides and proteins that are or have been post-translationally modified and include any sugar or other derivative(s) or conjugate(s) added to the backbone amino acid chain.

“Purified,” as used herein, means separated from many other compounds or entities. A compound or entity may be partially purified, substantially purified, or pure. A compound or entity is considered pure when it is removed from substantially all other compounds or entities, i.e., is preferably at least about 90%, more preferably at least about 91%, 92%, 93%, 94%, 95%, 96%, 97%, 98%, 99%, or greater than 99% pure. A partially or substantially purified compound or entity may be removed from at least 50%, at least 60%, at least 70%, or at least 80% of the material with which it is naturally found, e.g., cellular material such as cellular proteins and/or nucleic acids.

The term “recombinant” indicates that the material (e.g., a polynucleotide or a polypeptide) has been artificially or synthetically (non-naturally) altered by human intervention. The alteration can be performed on the material within or removed from, its natural environment, or native state. Specifically, e.g., a promoter sequence is “recombinant” when it is produced by the expression of a nucleic acid segment engineered by the hand of man. For example, a “recombinant nucleic acid” is one that is made by recombining nucleic acids, e.g., during cloning, DNA shuffling or other procedures, or by chemical or other mutagenesis; a “recombinant polypeptide” or “recombinant protein” is a polypeptide or protein which is produced by expression of a recombinant nucleic acid; and a “recombinant virus,” e.g., a recombinant AAV virus, is produced by the expression of a recombinant nucleic acid.

The term “regulatory element,” as used herein, refers to a region or regions of a nucleic acid sequence that regulates transcription. Exemplary regulatory elements include, but are not limited to, enhancers, post-transcriptional elements, transcriptional control sequences, and such like.

The term “RNA segment” refers to an RNA molecule that has been isolated free of total cellular RNA of a particular species. Therefore, RNA segments can refer to one or more RNA segments (either of native or synthetic origin) that have been isolated away from, or purified free from, other RNAs. Included within the term “RNA segment,” are RNA segments and smaller fragments of such segments.

The term “a sequence essentially as set forth in SEQ ID NO:X” means that the sequence substantially corresponds to a portion of SEQ ID NO:X and has relatively few nucleotides (or amino acids in the case of polypeptide sequences) that are not identical to, or a biologically functional equivalent of, the nucleotides (or amino acids) of SEQ ID NO:X. The term “biologically functional equivalent” is well understood in the art, and is further defined in detail herein. Accordingly, sequences that have about 85% to about 90%; or more preferably, about 91% to about 95%; or even more preferably, about 96% to about 99%; of nucleotides that are identical or functionally equivalent to one or more of the nucleotide sequences provided herein are particularly contemplated to be useful in the practice of the invention.

Suitable standard hybridization conditions for nucleic acids for use in the present invention include, for example, hybridization in 50% formamide, 5×Denhardt's solution, 5×SSC, 25 mM sodium phosphate, 0.1% SDS and 100 μg/mL of denatured salmon sperm DNA at 42° C. for 16 hr followed by 1 hr sequential washes with 0.1×SSC, 0.1% SDS solution at 60° C. to remove the desired amount of background signal. Lower stringency hybridization conditions for the present invention include, for example, hybridization in 35% formamide, 5×Denhardt's solution, 5×SSC, 25 mM sodium phosphate, 0.1% SDS and 100 μg/mL denatured salmon sperm DNA or E. coli DNA at 42° C. for 16 hr followed by sequential washes with 0.8×SSC, 0.1% SDS at 55° C. Those of ordinary skill in the art will recognize that such hybridization conditions can be readily adjusted to obtain the desired level of stringency for a particular application.

As used herein, the term “structural gene” is intended to generally describe a polynucleotide, such as a gene, that is expressed to produce an encoded peptide, polypeptide, protein, ribozyme, catalytic RNA molecule, or antisense molecule.

The term “subject,” as used herein, describes an organism, including mammals such as primates, to which treatment with the compositions according to the present invention can be provided. Mammalian species that can benefit from the disclosed methods of treatment include, but are not limited to, apes; chimpanzees; orangutans; humans; monkeys; domesticated animals such as dogs and cats; livestock such as horses, cattle, pigs, sheep, goats, and chickens; and other animals such as mice, rats, guinea pigs, and hamsters.

The term “substantially complementary,” when used to define either amino acid or nucleic acid sequences, means that a particular subject sequence, for example, an oligonucleotide sequence, is substantially complementary to all or a portion of the selected sequence, and thus will specifically bind to a portion of an mRNA encoding the selected sequence. As such, typically the sequences will be highly complementary to the mRNA “target” sequence, and will have no more than about 1, about 2, about 3, about 4, about 5, about 6, about 7, about 8, about 9, or about 10 or so base mismatches throughout the complementary portion of the sequence. In many instances, it may be desirable for the sequences to be exact matches, i.e., be completely complementary to the sequence to which the oligonucleotide specifically binds, and therefore have zero mismatches along the complementary stretch. As such, highly complementary sequences will typically bind quite specifically to the target sequence region of the mRNA and will therefore be highly efficient in reducing, and/or even inhibiting the translation of the target mRNA sequence into polypeptide product.

Substantially complementary nucleic acid sequences will be greater than about 80 percent complementary (or “% exact-match”) to a corresponding nucleic acid target sequence to which the nucleic acid specifically binds, and will, more preferably be greater than about 85 percent complementary to the corresponding target sequence to which the nucleic acid specifically binds. In certain aspects, as described above, it will be desirable to have even more substantially complementary nucleic acid sequences for use in the practice of the invention, and in such instances, the nucleic acid sequences will be greater than about 90 percent complementary to the corresponding target sequence to which the nucleic acid specifically binds, and may in certain embodiments be greater than about 95 percent complementary to the corresponding target sequence to which the nucleic acid specifically binds, and even up to and including about 96%, about 97%, about 98%, about 99%, and even about 100% exact match complementary to all or a portion of the target sequence to which the designed nucleic acid specifically binds.

Percent similarity or percent complementary of any of the disclosed nucleic acid sequences may be determined, for example, by comparing sequence information using the GAP computer program, version 6.0, available from the University of Wisconsin Genetics Computer Group (UWGCG). The GAP program utilizes the alignment method of Needleman and Wunsch (1970). Briefly, the GAP program defines similarity as the number of aligned symbols (i.e., nucleotides or amino acids) that are similar, divided by the total number of symbols in the shorter of the two sequences. The preferred default parameters for the GAP program include: (1) a unary comparison matrix (containing a value of 1 for identities and 0 for non-identities) for nucleotides, and the weighted comparison matrix of Gribskov and Burgess (1986), (2) a penalty of 3.0 for each gap and an additional 0.10 penalty for each symbol in each gap; and (3) no penalty for end gaps.

As used herein, the term “substantially free” or “essentially free” in connection with the amount of a component preferably refers to a composition that contains less than about 10 weight percent, preferably less than about 5 weight percent, and more preferably less than about 1 weight percent of a compound. In preferred embodiments, these terms refer to less than about 0.5 weight percent, less than about 0.1 weight percent, or less than about 0.01 weight percent.

As used herein, the term “structural gene” is intended to generally describe a polynucleotide, such as a gene, that is expressed to produce an encoded peptide, polypeptide, protein, ribozyme, catalytic RNA molecule, or antisense molecule.

The term “subject,” as used herein, describes an organism, including mammals such as primates, to which treatment with the compositions according to the present invention can be provided. Mammalian species that can benefit from the disclosed methods of treatment include, but are not limited to, humans, non-human primates such as apes; chimpanzees; monkeys, and orangutans, domesticated animals, including dogs and cats, as well as livestock such as horses, cattle, pigs, sheep, and goats, or other mammalian species including, without limitation, mice, rats, guinea pigs, rabbits, hamsters, and the like.

The terms “substantially corresponds to,” “substantially homologous,” or “substantial identity,” as used herein, denote characteristics of a nucleic acid or an amino acid sequence, wherein a selected nucleic acid sequence or a selected amino acid sequence has at least about 70 or about 75 percent sequence identity as compared to a selected reference nucleic acid or amino acid sequence. More typically, the selected sequence and the reference sequence will have at least about 76, 77, 78, 79, 80, 81, 82, 83, 84 or even 85 percent sequence identity, and more preferably, at least about 86, 87, 88, 89, 90, 91, 92, 93, 94, or 95 percent sequence identity. More preferably still, highly homologous sequences often share greater than at least about 96, 97, 98, or 99 percent sequence identity between the selected sequence and the reference sequence to which it was compared.

As used herein, “synthetic” shall mean that the material is not of a human or animal origin.

“Targeting moiety” is any factor that may facilitate targeting of a specific site by a particle. For example, the targeting moiety may be a chemical targeting moiety, a physical targeting moiety, a geometrical targeting moiety, or a combination thereof. The chemical targeting moiety may be a chemical group or molecule on a surface of the particle; the physical targeting moiety may be a specific physical property of the particle, such as a surface such or hydrophobicity; the geometrical targeting moiety includes a size and a shape of the particle. Further, the chemical targeting moiety may be a dendrimer, an antibody, an aptamer, which may be a thioaptamer, a ligand, an antibody, or a biomolecule that binds a particular receptor on the targeted site. A physical targeting moiety may be a surface charge. The charge may be introduced during the fabrication of the particle by using a chemical treatment such as a specific wash. For example, immersion of porous silica or oxidized silicon surface into water may lead to an acquisition of a negative charge on the surface. The surface charge may be also provided by an additional layer or by chemical chains, such as polymer chains, on the surface of the particle. For example, polyethylene glycol chains may be a source of a negative charge on the surface. Polyethylene glycol chains may be coated or covalently coupled to the surface using methods known to those of ordinary skill in the art.

The term “therapeutically-practical period” means the period of time that is necessary for one or more active agents to be therapeutically effective. The term “therapeutically-effective” refers to reduction in severity and/or frequency of one or more symptoms, elimination of one or more symptoms and/or underlying cause, prevention of the occurrence of symptoms and/or their underlying cause, and the improvement or a remediation of damage.

A “therapeutic agent” may be any physiologically or pharmacologically active substance that may produce a desired biological effect in a targeted site in a subject. The therapeutic agent may be a chemotherapeutic agent, an immunosuppressive agent, a cytokine, a cytotoxic agent, a nucleolytic compound, a radioactive isotope, a receptor, and a pro-drug activating enzyme, which may be naturally occurring, produced by synthetic or recombinant methods, or a combination thereof. Drugs that are affected by classical multidrug resistance, such as vinca alkaloids (e.g., vinblastine and vincristine), the anthracyclines (e.g., doxorubicin and daunorubicin), RNA transcription inhibitors (e.g., actinomycin-D) and microtubule stabilizing drugs (e.g., paclitaxel) may have particular utility as the therapeutic agent. Cytokines may be also used as the therapeutic agent. Examples of such cytokines are lymphokines, monokines, and traditional polypeptide hormones. A cancer chemotherapy agent may be a preferred therapeutic agent. For a more detailed description of anticancer agents and other therapeutic agents, those skilled in the art are referred to any number of instructive manuals including, but not limited to, the Physician's Desk Reference and Hardman and Limbird (2001).

As used herein, a “transcription factor recognition site” and a “transcription factor binding site” refer to a polynucleotide sequence(s) or sequence motif(s), which are identified as being sites for the sequence-specific interaction of one or more transcription factors, frequently taking the form of direct protein-DNA binding. Typically, transcription factor binding sites can be identified by DNA footprinting, gel mobility shift assays, and the like, and/or can be predicted based on known consensus sequence motifs, or by other methods known to those of ordinary skill in the art.

“Transcriptional regulatory element” refers to a polynucleotide sequence that activates transcription alone or in combination with one or more other nucleic acid sequences. A transcriptional regulatory element can, for example, comprise one or more promoters, one or more response elements, one or more negative regulatory elements, and/or one or more enhancers.

“Transcriptional unit” refers to a polynucleotide sequence that comprises at least a first structural gene operably linked to at least a first cis-acting promoter sequence and optionally linked operably to one or more other cis-acting nucleic acid sequences necessary for efficient transcription of the structural gene sequences, and at least a first distal regulatory element as may be required for the appropriate tissue-specific and developmental transcription of the structural gene sequence operably positioned under the control of the promoter and/or enhancer elements, as well as any additional cis-sequences that are necessary for efficient transcription and translation (e.g., polyadenylation site(s), mRNA stability controlling sequence(s), etc.

As used herein, the term “transformation” is intended to generally describe a process of introducing an exogenous polynucleotide sequence (e.g., a viral vector, a plasmid, or a recombinant DNA or RNA molecule) into a host cell or protoplast in which the exogenous polynucleotide is incorporated into at least a first chromosome or is capable of autonomous replication within the transformed host cell. Transfection, electroporation, and “naked” nucleic acid uptake all represent examples of techniques used to transform a host cell with one or more polynucleotides.

As used herein, the term “transformed cell” is intended to mean a host cell whose nucleic acid complement has been altered by the introduction of one or more exogenous polynucleotides into that cell.

“Treating” or “treatment of” as used herein, refers to providing any type of medical or surgical management to a subject. Treating can include, but is not limited to, administering a composition comprising a therapeutic agent to a subject. “Treating” includes any administration or application of a compound or composition of the invention to a subject for purposes such as curing, reversing, alleviating, reducing the severity of, inhibiting the progression of, or reducing the likelihood of a disease, disorder, or condition or one or more symptoms or manifestations of a disease, disorder, or condition. In certain aspects, the compositions of the present invention may also be administered prophylactically, i.e., before development of any symptom or manifestation of the condition, where such prophylaxis is warranted. Typically, in such cases, the subject will be one that has been diagnosed for being “at risk” of developing such a disease or disorder, either as a result of familial history, medical record, or the completion of one or more diagnostic or prognostic tests indicative of a propensity for subsequently developing such a disease or disorder.

The term “vector,” as used herein, refers to a nucleic acid molecule (typically comprised of DNA) capable of replication in a host cell and/or to which another nucleic acid segment can be operatively linked so as to bring about replication of the attached segment. A plasmid, cosmid, or a virus is an exemplary vector.

In certain embodiments, it will be advantageous to employ one or more nucleic acid segments of the present invention in combination with an appropriate detectable marker (i.e., a “label,”), such as in the case of employing labeled polynucleotide probes in determining the presence of a given target sequence in a hybridization assay. A wide variety of appropriate indicator compounds and compositions are known in the art for labeling oligonucleotide probes, including, without limitation, fluorescent, radioactive, enzymatic or other ligands, such as avidin/biotin, etc., which are capable of being detected in a suitable assay. In particular embodiments, one may also employ one or more fluorescent labels or an enzyme tag such as urease, alkaline phosphatase or peroxidase, instead of radioactive or other environmentally less-desirable reagents. In the case of enzyme tags, colorimetric, chromogenic, or fluorogenic indicator substrates are known that can be employed to provide a method for detecting the sample that is visible to the human eye, or by analytical methods such as scintigraphy, fluorimetry, spectrophotometry, and the like, to identify specific hybridization with samples containing one or more complementary or substantially complementary nucleic acid sequences. In the case of so-called “multiplexing” assays, where two or more labeled probes are detected either simultaneously or sequentially, it may be desirable to label a first oligonucleotide probe with a first label having a first detection property or parameter (for example, an emission and/or excitation spectral maximum), which also labeled a second oligonucleotide probe with a second label having a second detection property or parameter that is different (i.e., discreet or discernible from the first label. The use of multiplexing assays, particularly in the context of genetic amplification/detection protocols are well-known to those of ordinary skill in the molecular genetic arts.

Biological Functional Equivalents

Modification and changes may be made in the structure of the nucleic acids, or to the vectors comprising them, as well as to mRNAs, polypeptides, or therapeutic agents encoded by them and still obtain functional systems that contain one or more therapeutic agents with desirable characteristics. As mentioned above, it is often desirable to introduce one or more mutations into a specific polynucleotide sequence. In certain circumstances, the resulting encoded polypeptide sequence is altered by this mutation, or in other cases, the sequence of the polypeptide is unchanged by one or more mutations in the encoding polynucleotide.

When it is desirable to alter the amino acid sequence of a polypeptide to create an equivalent, or even an improved, second-generation molecule, the amino acid changes may be achieved by changing one or more of the codons of the encoding DNA sequence, according to Table 2.

For example, certain amino acids may be substituted for other amino acids in a protein structure without appreciable loss of interactive binding capacity with structures such as, for example, antigen-binding regions of antibodies or binding sites on substrate molecules. Since it is the interactive capacity and nature of a protein that defines that protein's biological functional activity, certain amino acid sequence substitutions can be made in a protein sequence, and, of course, its underlying DNA coding sequence, and nevertheless obtain a protein with like properties. It is thus contemplated by the inventors that various changes may be made in the peptide sequences of the disclosed compositions or corresponding DNA sequences which encode said peptides without appreciable loss of their biological utility or activity.

TABLE 2

| | Amino Acids | Codons | | | | | |
|---|---|---|---|---|---|---|---|
| Alanine | Ala | GCA | GCC | GCG | GCU | | |
| Cysteine | Cys | UGC | UGU | | | | |
| Aspartic acid | Asp | GAC | GAU | | | | |
| Glutamic acid | Glu | GAA | GAG | | | | |
| Phenylalanine | Phe | UUC | UUU | | | | |
| Glycine | Gly | GGA | GGC | GGG | GGU | | |
| Histidine | His | CAC | CAU | | | | |
| Isoleucine | Ile | AUA | AUC | AUU | | | |
| Lysine | Lys | AAA | AAG | | | | |
| Leucine | Leu | UUA | UUG | CUA | CUC | CUG | CUU |
| Methionine | Met | AUG | | | | | |
| Asparagine | Asn | AAC | AAU | | | | |
| Proline | Pro | CCA | CCC | CCG | CCU | | |
| Glutamine | Gln | CAA | CAG | | | | |
| Arginine | Arg | AGA | AGG | CGA | CGC | CGG | CGU |
| Serine | Ser | AGC | AGU | UCA | UCC | UCG | UCU |
| Threonine | Thr | ACA | ACC | ACG | ACU | | |
| Valine | Val | GUA | GUC | GUG | GUU | | |
| Tryptophan | Trp | UGG | | | | | |
| Tyrosine | Tyr | UAC | UAU | | | | |

In making such changes, the hydropathic index of amino acids may be considered. The importance of the hydropathic amino acid index in conferring interactive biologic function on a protein is generally understood in the art (Kyte and Doolittle, 1982, incorporate herein by reference). It is accepted that the relative hydropathic character of the amino acid contributes to the secondary structure of the resultant protein, which in turn defines the interaction of the protein with other molecules, for example, enzymes, substrates, receptors, DNA, antibodies, antigens, and the like. Each amino acid has been assigned a hydropathic index based on its hydrophobicity and charge characteristics (Kyte and Doolittle, 1982). These values are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

It is known in the art that certain amino acids may be substituted by other amino acids having a similar hydropathic index or score and still result in a protein with similar biological activity, i.e. still obtain a biological functionally equivalent protein. In making such changes, the substitution of amino acids whose hydropathic indices are within ±2 is preferred, those within ±1 are particularly preferred, and those within ±0.5 are even more particularly preferred. It is also understood in the art that the substitution of like amino acids can be made effectively based on hydrophilicity. U.S. Pat. No. 4,554,101 (specifically incorporated herein in its entirety by express reference thereto), states that the greatest local average hydrophilicity of a protein, as governed by the hydrophilicity of its adjacent amino acids, correlates with a biological property of the protein.

As detailed in U.S. Pat. No. 4,554,101, the following hydrophilicity values have been assigned to amino acid residues: arginine (+3.0); lysine (+3.0); aspartate (+3.0±1); glutamate (+3.0±1); serine (+0.3); asparagine (+0.2); glutamine (+0.2); glycine (0); threonine (−0.4); proline (−0.5±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4). It is understood that an amino acid can be substituted for another having a similar hydrophilicity value and still obtain a biologically equivalent, and in particular, an immunologically equivalent protein. In such changes, the substitution of amino acids whose hydrophilicity values are within ±2 is preferred, those within ±1 are particularly preferred, and those within ±0.5 are even more particularly preferred.

As outlined above, amino acid substitutions are generally therefore based on the relative similarity of the amino acid side-chain substituents, for example, their hydrophobicity, hydrophilicity, charge, size, and the like. Exemplary substitutions that take one or more of the foregoing characteristics into consideration are well known to those of ordinary skill in the art, and include arginine and lysine; glutamate and aspartate; serine and threonine; glutamine and asparagine; and valine, leucine and isoleucine.

The section headings used throughout are for organizational purposes only and are not to be construed as limiting the subject matter described. All documents, or portions of documents, cited in this application (including, but not limited to, patents, patent applications, articles, books, and treatises) are expressly incorporated herein in their entirety by express reference thereto. In the event that one or more of the incorporated literature and similar materials defines a term in a manner that contradicts the definition of that term in this application, this application controls.

### EXAMPLES

The following examples are included to demonstrate illustrative embodiments of the invention. It should be appreciated by those of ordinary skill in the art that the techniques disclosed in these examples represent techniques discovered to function well in the practice of the invention, and thus can be considered to constitute preferred modes for its practice. However, those of ordinary skill in the art should, in light of the present disclosure appreciate that many changes can be made in the specific embodiments which are disclosed and still obtain a like or similar result without departing from the spirit and scope of the invention.

### Example 1

Although many clinical trials have been reported using NY-ESO-1 peptide with Montanide ISA-51 plus CpG or poly (I:C), bot clinical and immune responses generally are weak or modest. One of many plausible reasons is that NY-ESO-1 peptide and TLR ligands [CpG or poly (I:C)] do not interact each other to form a complex or particle, leading to weak immune response. It was previously demonstrated that DC/TAT-TRP-2 vaccines could generate strong protective, but not therapeutic, immunity (Wang et al., 2002). In a phase I clinical trial, TAT-ESO-1 peptide was mixed with Montanide ISA-51 for vaccine, which generated only weak T cell response. During the course of this study, we discovered two potential problems: 1) TAT-ESO-1 could not form a stable complex with Montanide ISA-51 probably due to its positive charges in its N-terminus; 2) TAT-ESO-1 formed a precipitation when it mixed with CpG. Based on the electric charges and hydrophobic properties, we designed and developed a novel technology of self-assembled CPP-T-cell peptide nanoparticles (PEP-NANO) with TLR ligands [CpG, MPLA and poly (I:C), CMI for short], as schematically presented in FIG. 2A and FIG. 2B. Amphiphobic or amphipathic CPP-therapeutic peptides, consisting of CPP such as TAT with positively charged peptide and covalently linked to a therapeutic peptide such as NY-ESO-1 (SLLMWITQCFLPV) (SEQ ID NO:1) and TRP-2 (SYVDFFVWL) (SEQ ID NO:2) (generally hydrophobic), form nanoparticles with negatively charged CpG and/or poly (I:C) through electric interactions, while with MPLA through hydrophobicity inside the particles.

### Example 2

Self-Assembly and Characterization of TAT-TRP2 Peptide Nanoparticles with TLR Ligands or their Combinations

TAT-TRP2, TAT-ESO-1 and CpG were first completely dissolved in sterile ultrapure water as stock solution at a concentration of 10 mg/mL. MPLA and poly I:C were completely dissolved in sterile ultrapure water at a concentration of 1 mg/mL. To prepare the TAT-peptide vaccine nanoparticles, CpG, MPLA, and poly I:C of indicated volume (shown in Table 3) were mix thoroughly in ultrapure water or buffer by intensive vortex.

TABLE 3
CONCENTRATIONS AND COMBINATIONS OF TLR LIGANDS
Ligands Combinations

| | | Name | | | | |
|---|---|---|---|---|---|---|
| | | Peptide | TLR9 | TLR3 | TLR4 | TLR7/8 |
| | | TAT-TRP2 | CpG1826 | Poly (I:C) | MPLA | R848 |
| | | Stock Conc. | | | | |
| | | 10 mg/mL | 10 mg/mL | 1 mg/mL | 1 mg/mL | 1 mg/mL |
| | | Cell Working Conc. | | | | |
| | | / | 2 ug/mL | 2 ug/mL | 1 ug/mL | 1 ug/mL |
| | | Animal Working Conc. | | | | |
| | | 100 ug/mouse | 10-50 ug/mouse | 10-100 ug/mouse | 2-20 ug/mouse | 10-100 ug/mouse |
| CpG | 1 | 10 | 1.67 | 0 | 4 | 0 |
| MPLA | 2 | 10 | 2.06 | 0 | 4 | 0 |
| | 3 | 10 | 4.11 | 0 | 4 | 0 |
| | 4 | 10 | 9.07 | 0 | 4 | 0 |
| CpG | 5 | 10 | 2.06 | 5 | 0 | 0 |
| Poly(IC) | 6 | 10 | 2.06 | 10 | 0 | 0 |
| | 7 | 10 | 2.06 | 20 | 0 | 0 |
| | 8 | 10 | 2.06 | 60 | 0 | 0 |
| Poly(IC) | 9 | 10 | 0 | 5 | 4 | 0 |
| MPLA | 10 | 10 | 0 | 10 | 4 | 0 |
| | 11 | 10 | 0 | 20 | 4 | 0 |
| | 12 | 10 | 0 | 60 | 4 | 0 |
| CpG | 13 | 10 | 2.06 | 0 | 0 | 5 |
| R848 | 14 | 10 | 2.06 | 0 | 0 | 10 |
| | 15 | 10 | 2.06 | 0 | 0 | 20 |
| | 16 | 10 | 2.06 | 0 | 0 | 60 |
| CpG | 17 | 10 | 2.06 | 5 | 4 | 0 |
| MPLA | 18 | 10 | 2.06 | 10 | 4 | 0 |
| Poly(IC) | 19 | 10 | 2.06 | 20 | 4 | 0 |
| | 20 | 10 | 2.06 | 60 | 4 | 0 |
| CpG | 21 | 10 | 2.06 | 0 | 4 | 5 |
| MPLA | 22 | 10 | 2.06 | 0 | 4 | 10 |
| R848 | 23 | 10 | 2.06 | 0 | 4 | 20 |
| | 24 | 10 | 2.06 | 0 | 4 | 60 |

Self-assembly of the TAT-peptide nanoparticles was triggered by dropwise addition of TAT-peptide with ultrasonication in iced water bath within 1 min. TAT-TRP2 exhibits the hydrophilicity/positive charge and hydrophobicity, and also facilitates its electrostatic interactions with CpG ODN. The hydrophobic C-terminus of TRP2 peptide forms a hydrophobic core together with the MPLA via hydrophobic interactions. Electrostatic and hydrophobic interactions drive the self-assembly of TAT-TRP2 with double or triple TLR agonists to form sphere-shaped TAT-TRP2-CM nanoparticles, as demonstrated by AFM analysis for both height and DMT modulus distribution over a representative cross section (red line). By contrast, TRP2 peptide without TAT modification failed to form complex with CpG-MPLA and formed long fibers.

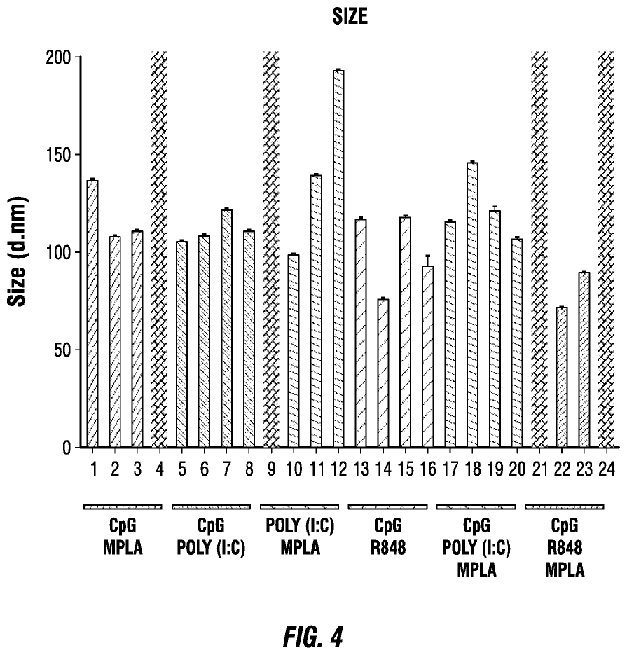

In order to formulate a stable and high-efficient peptide vaccine nanoparticles, we optimized the formulations using different ratios of TAT-TRP2 peptide:TLR ligands as shown in Table 3, and characterized the size of each nanoparticles (80-150 nm) of TAT-TRP2 with different combinations of TLR ligands (FIG. 4). Grey bars indicate unstable/polydispersed complexes with large PDI (PDI>0.5).

### Example 3

Zeta Potential of CPP-Peptide Nanoparticles

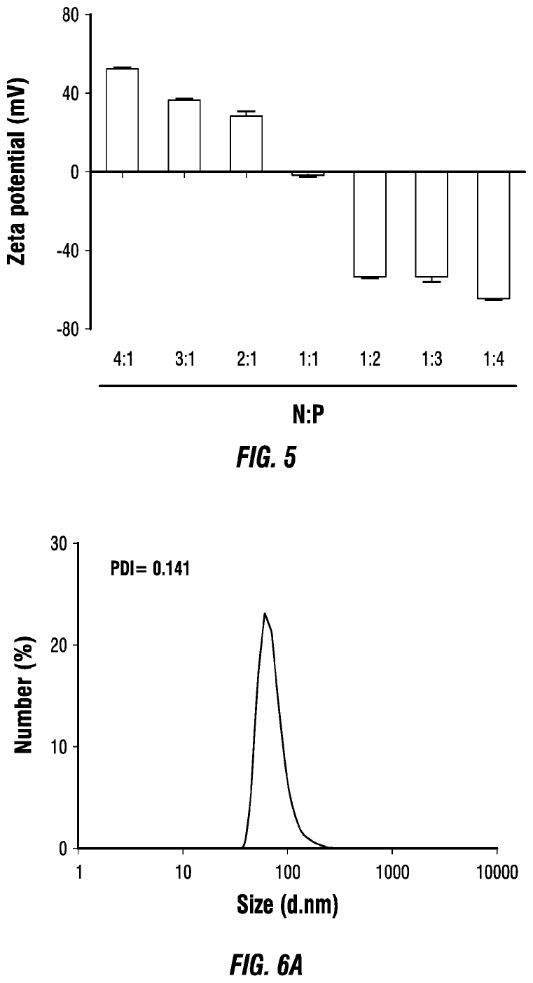

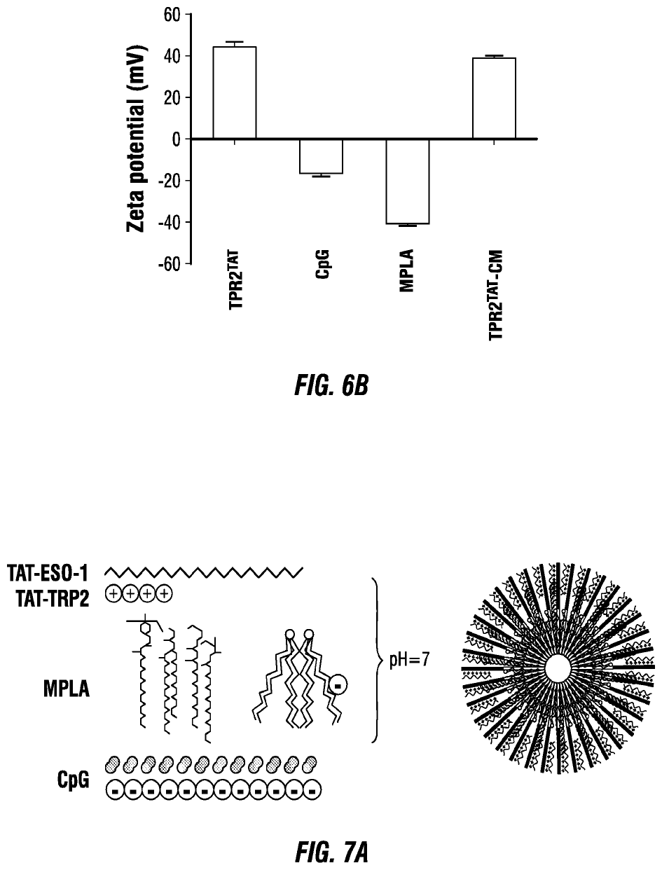

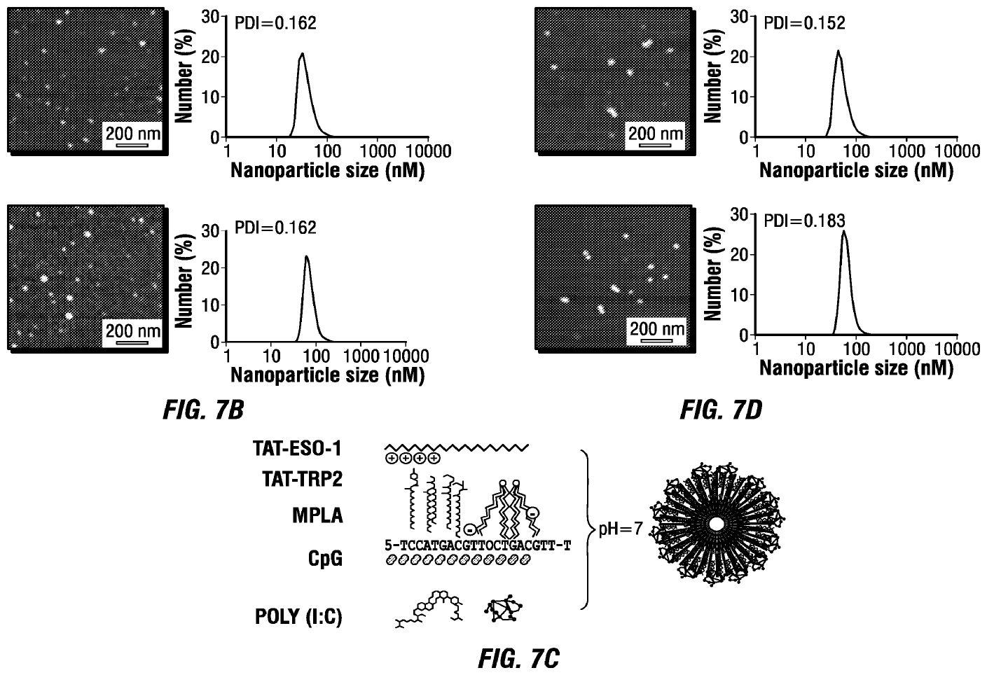

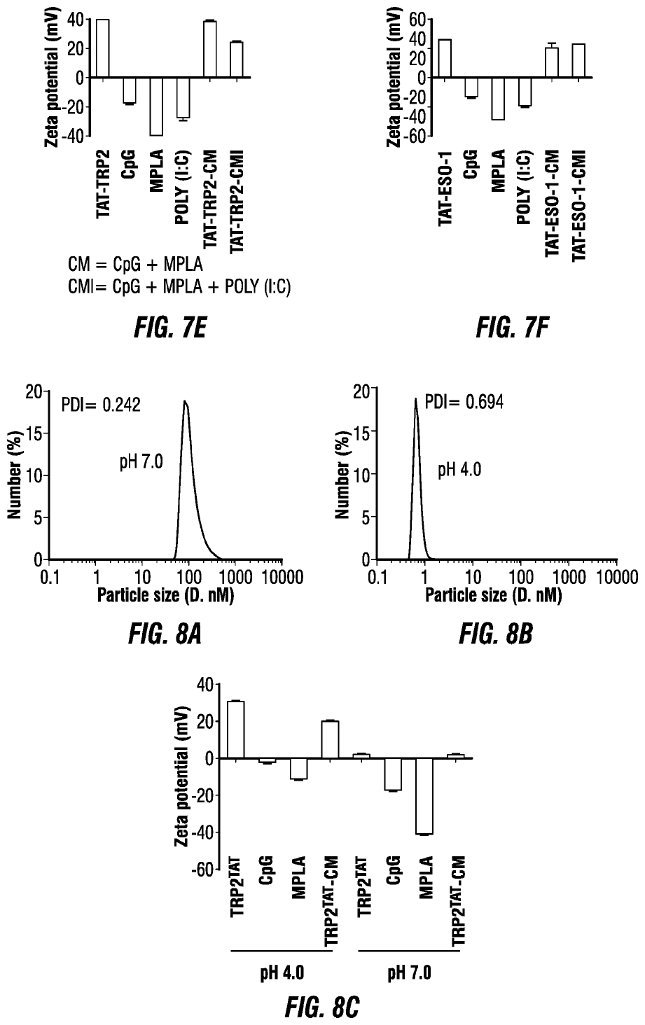

To determine the surface charges of nanoparticles, the zeta potential was measured for complexes constituted with various nitrogen (+) over phosphate (−) (N/P) ratios (N=nitrogen from amino acid residues; P=CpG ODN phosphate groups). The zeta potential of TAT-TRP2-CM nanoparticles was changed with different ratios of N/P (FIG. 5). Condition #2 for TAT-TRP2-CM and #18 for TAT-TRP2-CMI (Table 3 and FIG. 4) were applied for future studies. FIG. 6A and FIG. 6B show the nanoparticle size of TAT-TRP2-CM, and zeta potential of TAT-TRP2, CpG, MPLA and TAT-TRP2-CM. Similar results were obtained using TAT-ESO-1 with CpG and MPLA (CM) and CpG, MPLA and poly (I:C) (CMI) (FIG. 7A, FIG. 7B, FIG. 7C, FIG. 7D, FIG. 7E, and FIG. 7F).

### Example 4

CPP-Peptide Nanoparticle Formation & Size are Both pH-Dependent

It was demonstrated that the nanoparticles and zeta potential of TAT-TRP2-CM at pH7.0 were disrupted and changed at pH 4.0 (FIG. 8A, FIG. 8B, and FIG. 8C).

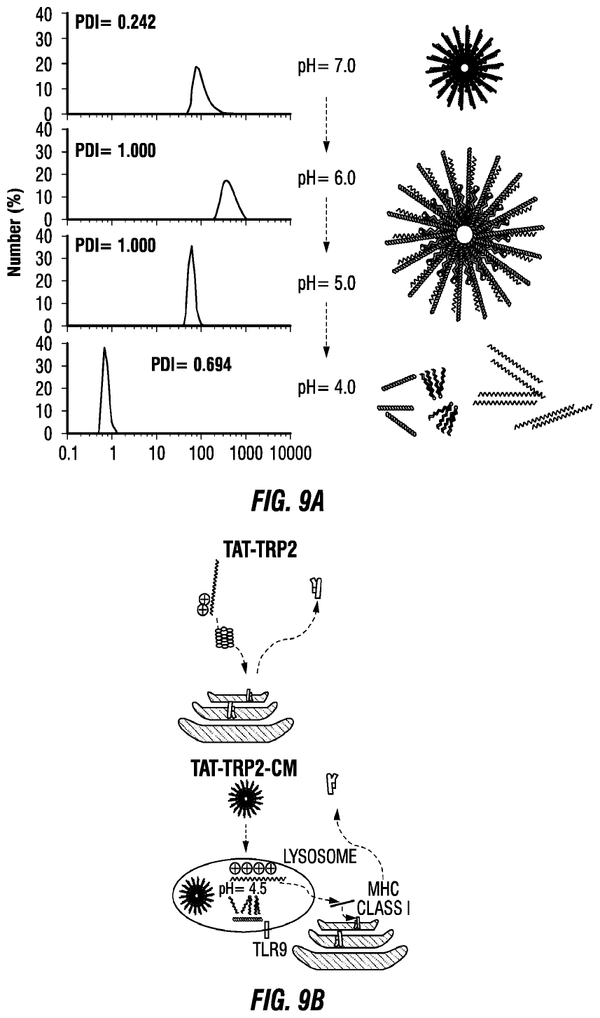

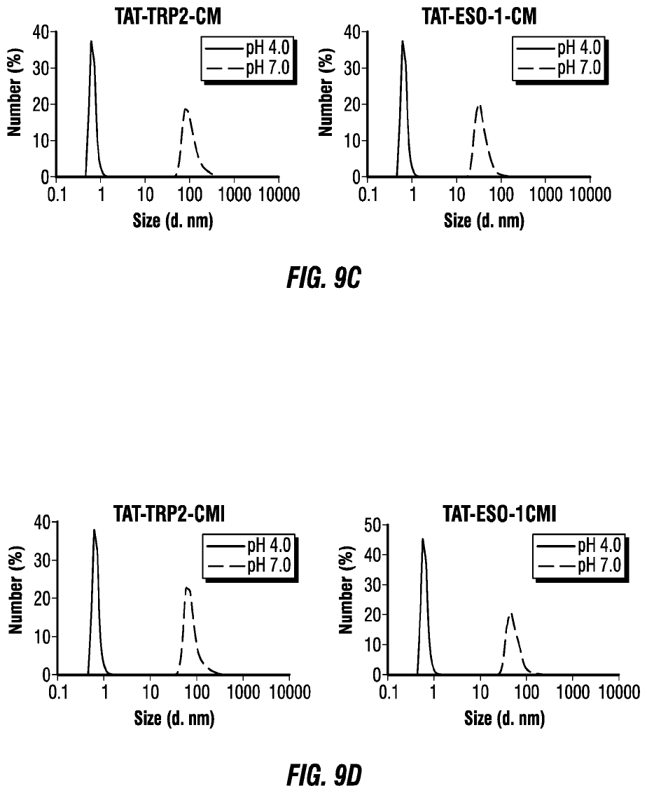

To further characterize the self-assembly and nanoparticle sizes under different pH 4-7, we found that the self-assembly and nanoparticles were disrupted in different pH values. Under pH 7.0, nanoparticles of TAT-TRP2-CM were tight and 100 nm in size, but increased the size at pH 6.0, decreased at pH 5.0. The nanoparticles of TAT-TRP2-CM were completely disrupted and separated (FIG. 9A). Based on these results, we reasoned that TAT-TRP2-CM nanoparticles are uptaken by APCs into endosomes/lysosomes upon phagocytosis, where the nanoparticles are disrupted in pH 4-5. Acidification within endo/lysosomal compartments increase the positive charge of TAT-TRP2 and neutralize the CpG ODN. TAT-TRP2 peptides are then released into cytoplasmic and presented by MEW class I or II molecules in ERs, while TLR ligands binds to TLRs to trigger innate immune response and produce cytokines, thus enhancing antigen presentation and T cell activation (FIG. 9B). By contrast, TAT-TRP2 alone enters APCs through the cell-penetrating property, and is presented by APCs to T cells, without innate immune response and cytokine production. It was further shown that the pH-dependent property was applied to TAT-TRP2-CM, TAT-TRP2-CMI, TAT-ESO-1-CM and TAT-ESO-1-CMI (FIG. 9C).

### Example 5

CPP-Peptide Nanoparticles Triggering Innate Immune Response and Cytokine Production with Different TLR Ligand Combinations

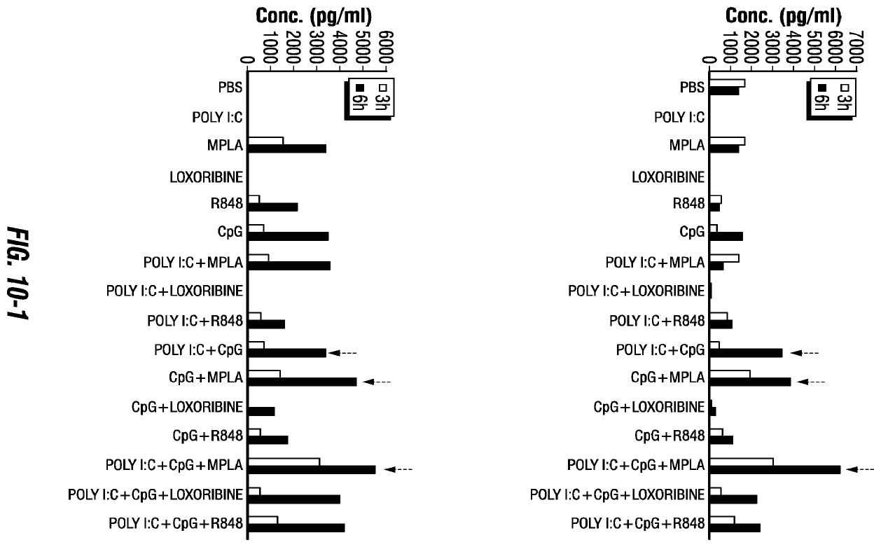

To identify the best combination of TLR ligands to stimulate innate immune response, we freshly isolated bone-marrow-derived DCs and then treated with different TLR ligands (single alone), double or triple combinations (FIG. 9A, FIG. 9B, FIG. 9C and FIG. 9D). After treatment of different TLR ligands or their combinations, cytokine (TNF-α, IL-6, IFN-α and IFN-β) production was determined in cell supernatants by ELISA. It was found that poly (I:C)/CpG, CpG/MPLA double combinations, and CpG/poly(I:C)/MPLA triple combination were better than other groups in triggering innate immune cytokine production. The CpG/poly(I:C)/MPLA triple combination is the strongest activator to induce cytokine production (FIG. 10).

### Example 6

CPP-Peptide Nanoparticle Formation and Sizes are pH-Dependent

Although many clinical trials have been reported using NY-ESO-1 peptide with Montanide ISA-51 plus CpG or poly (I:C), but clinical and immune responses generally are weak or modest. One of many plausible reasons is that NY-ESO-1 peptide and TLR ligands [CpG or poly (I:C)] do not interact each other to form a complex or particle, leading to weak immune response. It was previously demonstrated that DC/TAT-TRP-2 vaccines could generate strong protective, but not therapeutic, immunity (Wang et al., 2002). In a phase I clinical trial, TAT-ESO-1 peptide was mixed with Montanide ISA-51 for vaccine, which generated only weak T cell response. Overall, murine and human clinical studies show that current vaccine approaches fail to potent immune and clinical responses. The major issue is that cancer antigen peptide/protein and TLR ligands are not co-delivered into the same APCs. In most cases, only one of TLR ligands is used, rather than two or three ligands are co-delivered. In the following examples, we demonstrate that SAPEP-NANO technology, which utilize amphipathic CPP-therapeutic peptides, such as TAT-TRP2 or TAT-ESO-1, to form nanoparticles with negatively charged CpG and/or poly (I:C) through electric interactions, as well as with MPLA through hydrophobicity, can generate potent antitumor immunity in mouse models.

### Example 7

Generation of Robust Antitumor Immunity by DCs Loaded with TAT-TRP2 and TLR Ligand Nanoparticles

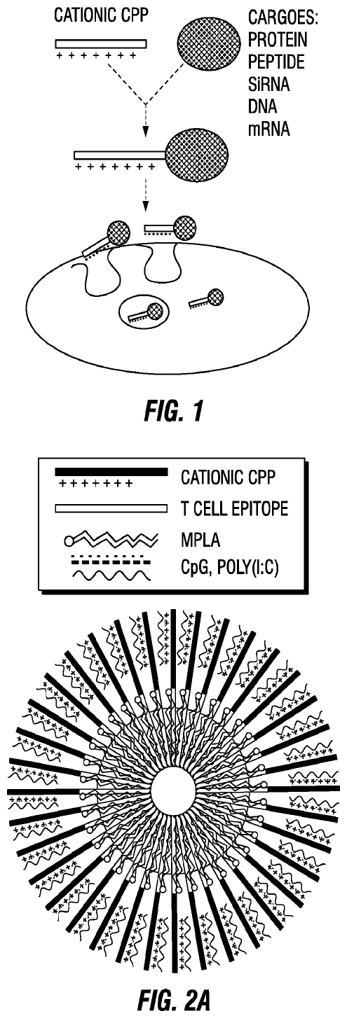

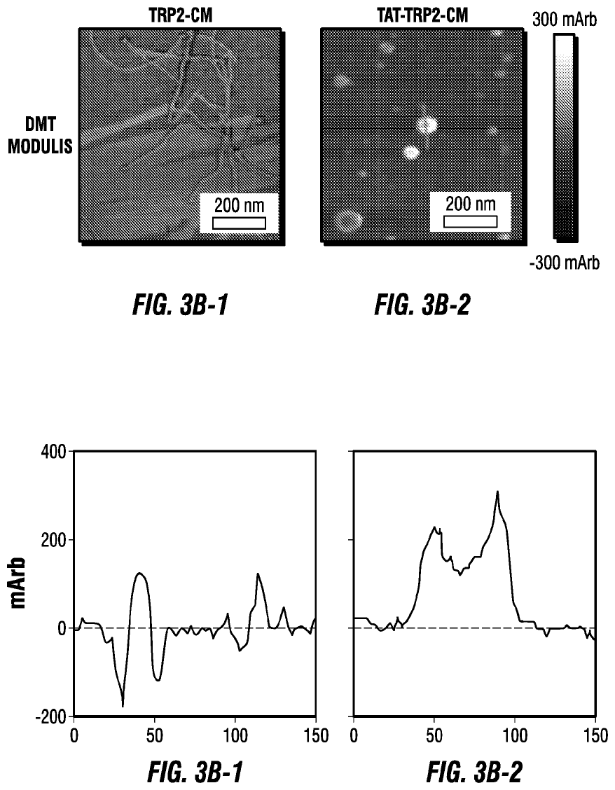

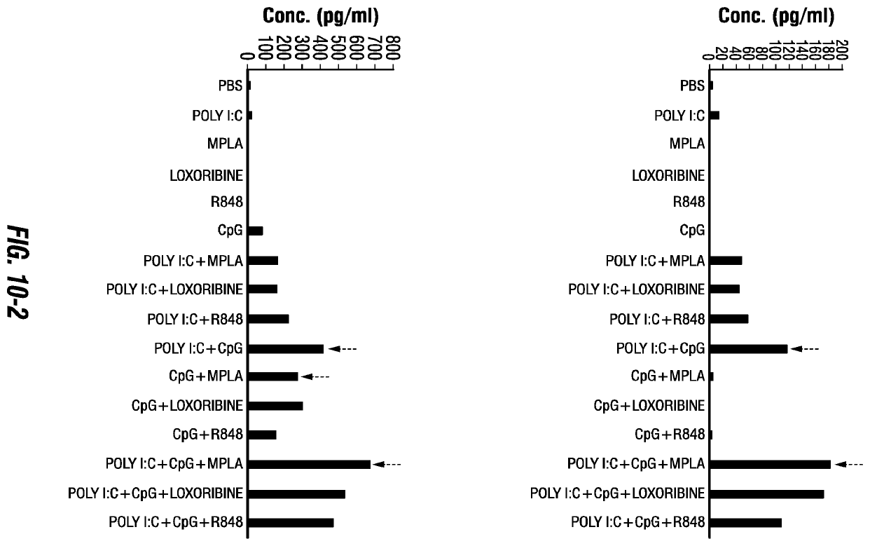

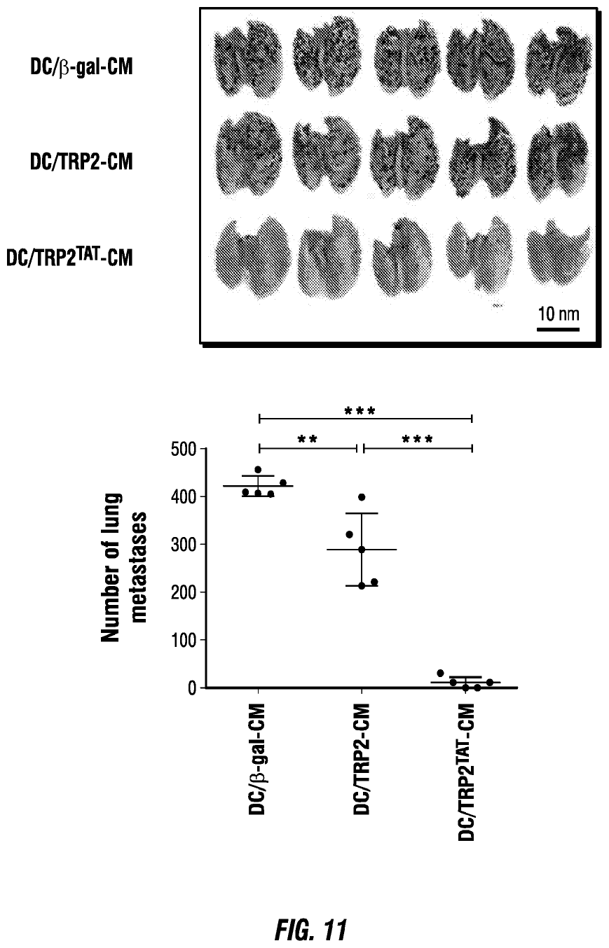

To enhance antitumor immunity, we hypothesized that TAT-TRP-2 peptide may form a complex with TLR ligands such as CpG and MPLA through physical properties (positive/negative charges, hydrophilic and hydrophobic) and induce therapeutic immunity. To test this prediction, we used a B16 mouse model and a tyrosinase-related protein-2 (TRP-2) peptide as our experimental system. TAT-TRP-2 (YGRKKRRQRRRSYVDFFVWL) (SEQ ID NO:17) peptide formed tight complexes with CpG/MPLA (TAT-TRP2-CM), while TRP2 peptide failed to form complexes with CpG/MPLA (TRP2-CM (FIG. 3A-1, FIG. 3A-2, FIG. 3B-1, FIG. 3B-2, FIG. 3C-1 and FIG. 3C-2). DCs loaded with TAT-TRP2-CM or TRP2-CM were prepared and intravenously injected into B16 tumor bearing mice. After 16 days, lung metastasis was examined in these treated mice and it was found that DC/TAT-TRP2-CM markedly inhibited the number of lung metastasis, while DC/TRP2-CM failed to inhibit the number of lung metastasis compared with DC/beta-gal-CM control group (FIG. 11).

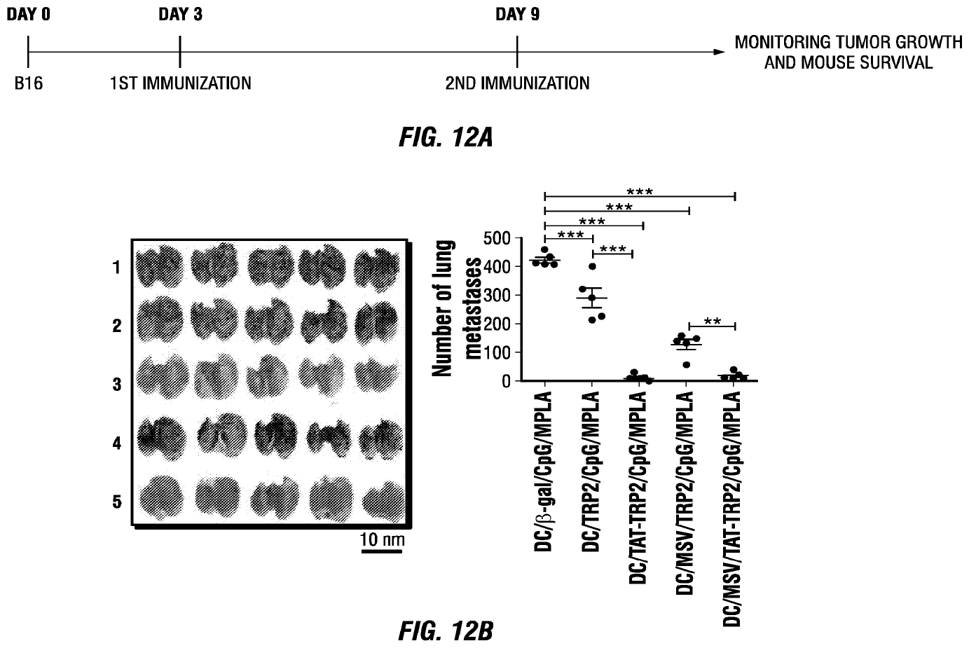

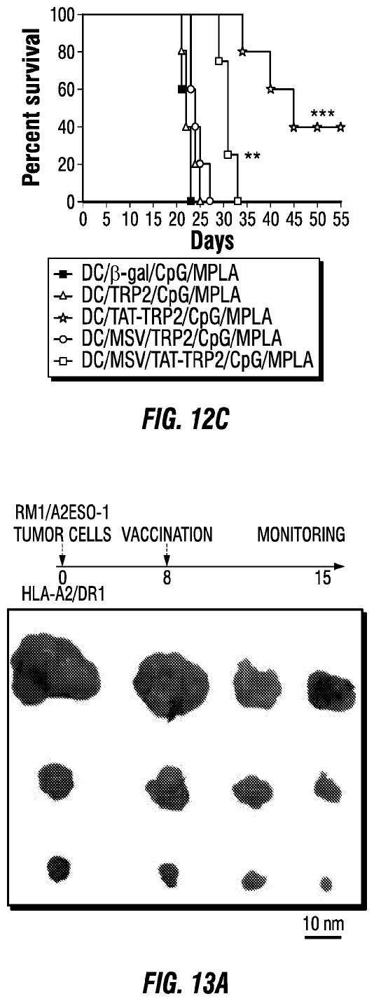

It was recently shown that multistage vehicle (MSV) nanotechnology can load peptide, CpG and MPLA into silicon particles, and induce strong immune response against B16 tumor cells Zhu et al., 2018). In order to compare DC/MSV vaccine with DC/PEP-NANO vaccine for their ability to induce antitumor immunity and survival, we prepared DC/control peptide (group #1), DC/TRP2/CpG/MPLA (#2) DC/TAT-TRP-2/CpG/MPLA (group #3) DC/MSV-TRP2/CpG/MPLA (#4) and DC/MSV-TAT-TRP-2/CpG/MPLA (#5), and then injected into tumor-bearing mice (FIG. 12A). Evidently, DC/TAT-TRP2/CpG/MPLA group (#3) and DC/MSV-TAT-TRP2/CpG/MPLA group (#5) could induce stronger therapeutic immunity and inhibit B16 lung metastasis, compared with DC/TRP-2/CpG/MPLA (#2) and DC/MSV/TRP-2/CpG/MPLA (#4) regardless of MSV (FIG. 12B), suggesting that TAT sequence, but MSV, is critically required for generating the strongest immune response. More importantly, we further showed that mice immunized with the DC/TAT-TRP-2/CpG/MPLA could survive much longer than those in DC/MSV-TAT-TRP-2/CpG/MPLA group, in which all mice died within 35 days after B16 tumor injection (FIG. 12C). Other vaccine groups (DC/beta-Gal/CpG/MPLA, DC/TRP2/CpG/MPLA and DC/MSV/TRP-2/CpG/MPLA) died within 25 days after B16 inoculation. These results suggested that TAT-TRP-2/CpG/MPLA vaccine was the best among the different vaccine groups tested.

### Example 8

Generation of Robust Antitumor Immunity by DCs Loaded with TAT-ESO-1 and TLR Ligand Nanoparticles

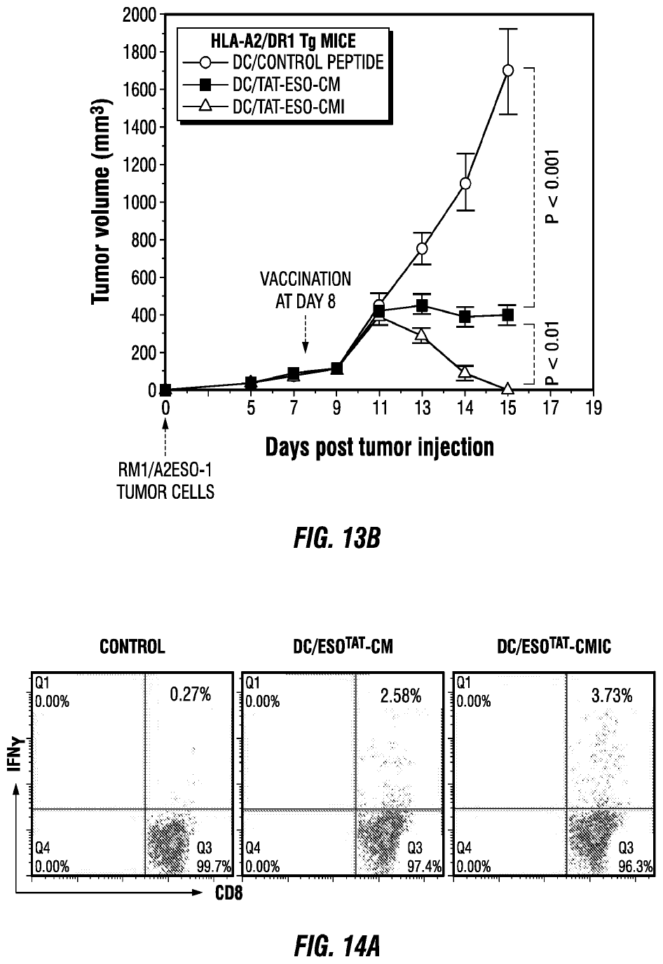

To investigate whether DCs loaded with TAT-ESO-1-CM or TAT-ESO-CMI nanoparticles could induce potent antitumor immunity against RM1/A2-ESO-1 tumor cells, experiments were performed with vaccination of DC/control peptide, DC/TAT-ESO-CM or DC/TAT-ESO-CMI. Tumor growth was monitored every two days. It was found that DC/TAT-ESO-CM vaccination markedly inhibited RM1/HLA-A2-NY-ESO-1 tumor growth, compared with control group (FIG. 13A and FIG. 13B). Importantly, DC/TAT-ESO-CMI showed much stronger immunity than DC/TAT-ESO-CM vaccine (FIG. 13A and FIG. 13B). Further analysis of immune cell response confirmed that the antigen-specific response in DC/TAT-ESO-CMI was better than DC/TAT-ESO-CM (FIG. 14A and FIG. 14B). These results suggested that both DC/TAT-ESO-CM and DC/TAT-ESO-CMI vaccines produced potent antitumor immunity.

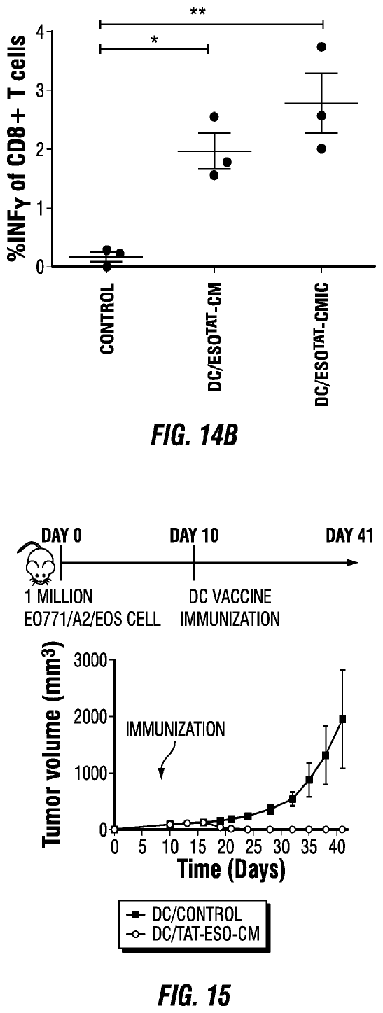

To further demonstrate whether DC/TAT-ESO-CM could induce therapeutic antitumor immunity in other tumor models, breast cancer E0771/A2-ESO tumor cells were used as a tumor model. It was shown that DC/TAT-ESO-CM vaccination completely inhibited tumor growth as compared to the control (FIG. 15).

### Example 9

Direct Immunization of TAT-ESO-1/TLR Nanoparticles without DCs Induces Strong and Therapeutic Antitumor Immunity

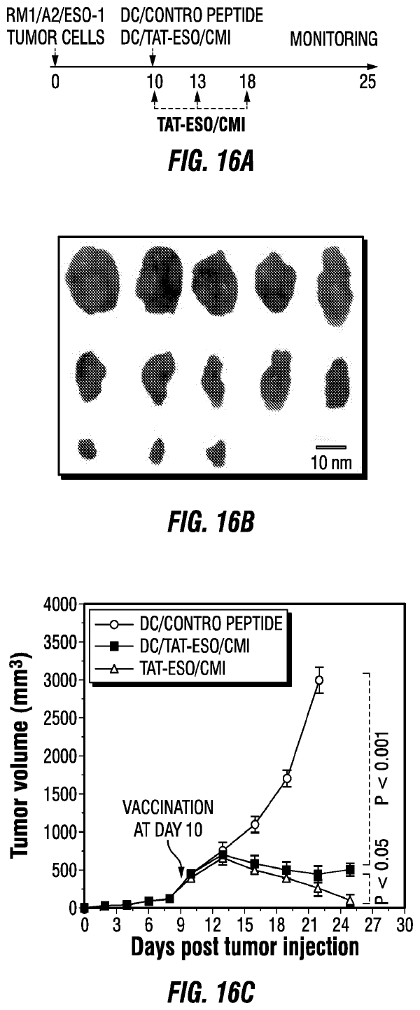

Although most studies for vaccines use DCs loaded with antigenic peptides, MSV particles, or a combination of CPP-peptides and TLR nanoparticles, such processes are complicated and very labor-intensive, particularly for clinical trials. Thus, the inventors hypothesized whether CPP-peptide/TLR nanoparticles could be directly used for vaccination to generate potent antitumoral immunity. To test this possibility, tumor-bearing mice were immunized three times with TAT-ESO-CMI, and compared to mice immunized once with DC/TAT-ESO-CMI. The results are shown in FIG. 16A. Direct vaccination with TAT-ESO-CMI markedly inhibited tumor growth, and to a degree much higher than DC/TAT-ESO-CMI (FIG. 16B and FIG. 16C). In contrast, and as expected, rapid tumor growth was observed in the control group.

### Example 10

### TAT-CT83 Peptide Vaccines

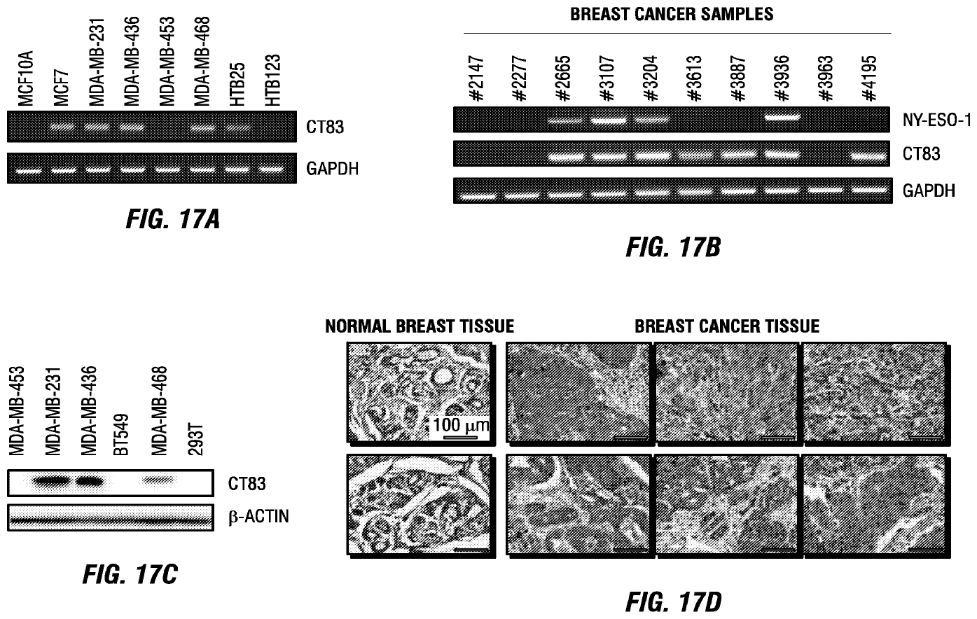

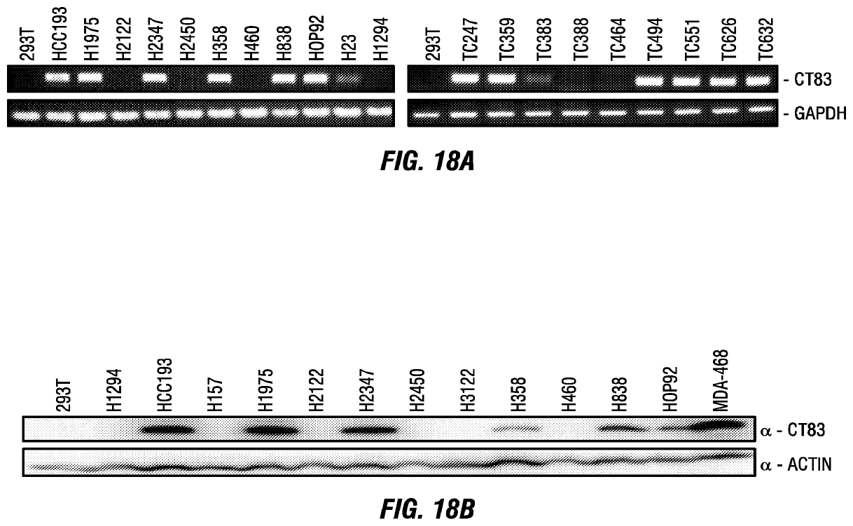

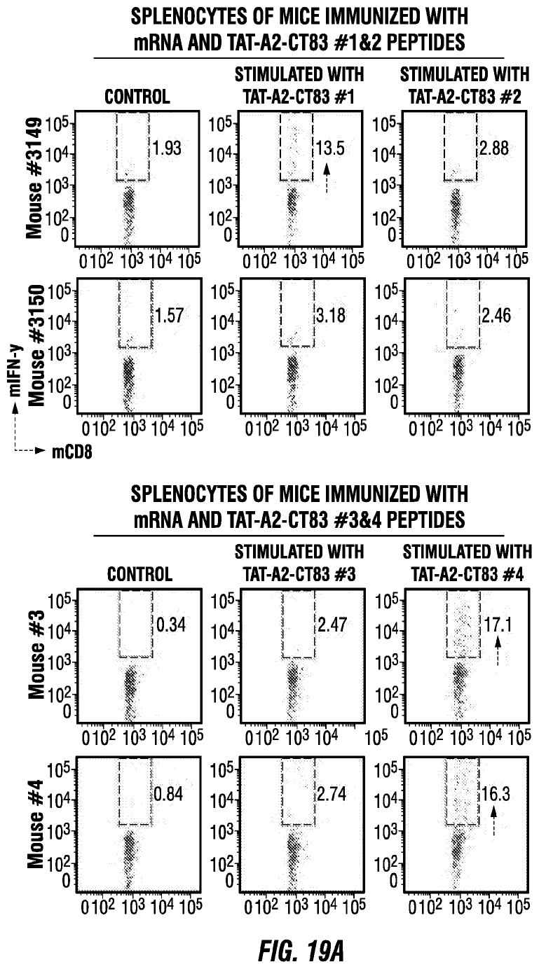

CT83 (also known as CXORF61 and KKLC1) has been shown to be highly expressed in human lung and breast cancer (FIG. 17A-FIG. 17D, FIG. 18A and FIG. 18B), consistent with previous reports (Fukuyama et al., 2006; Paret et al., 2015). Thus, it is likely that CT83 can serve as an immune target for cancer vaccine and immunotherapy.

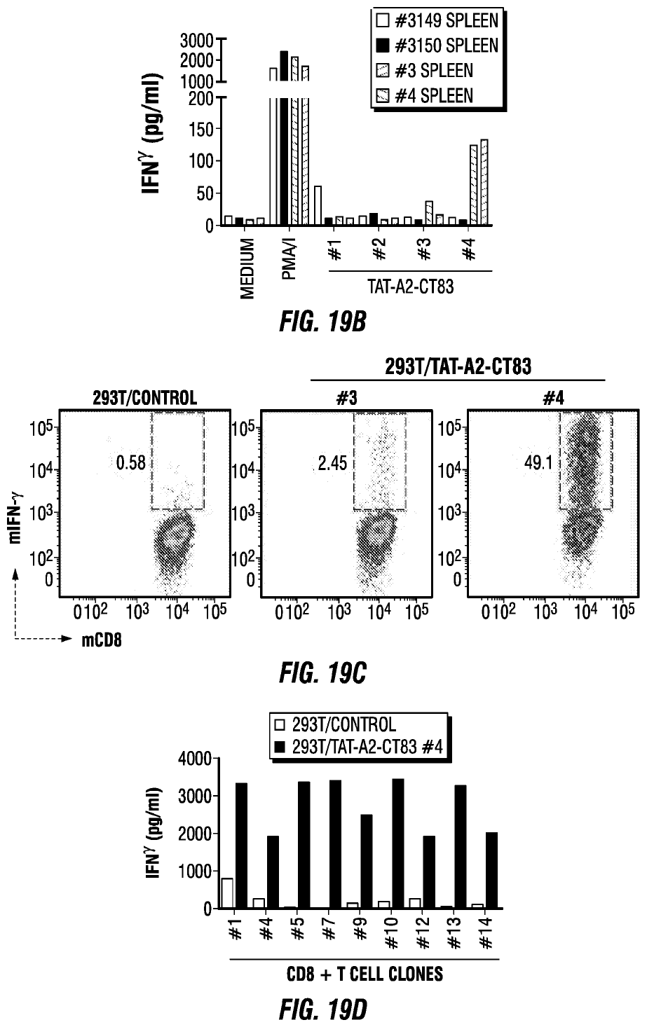

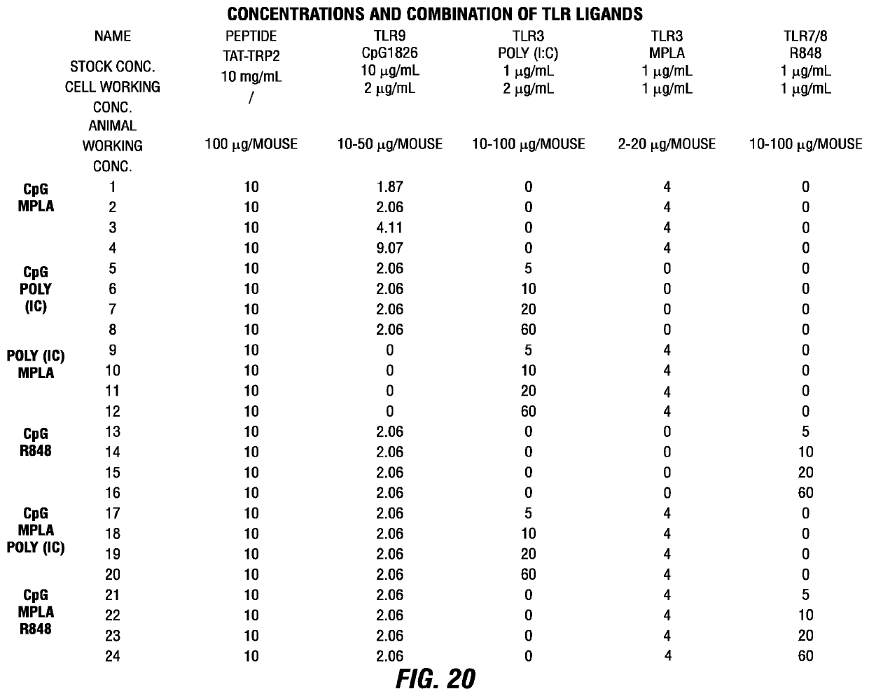

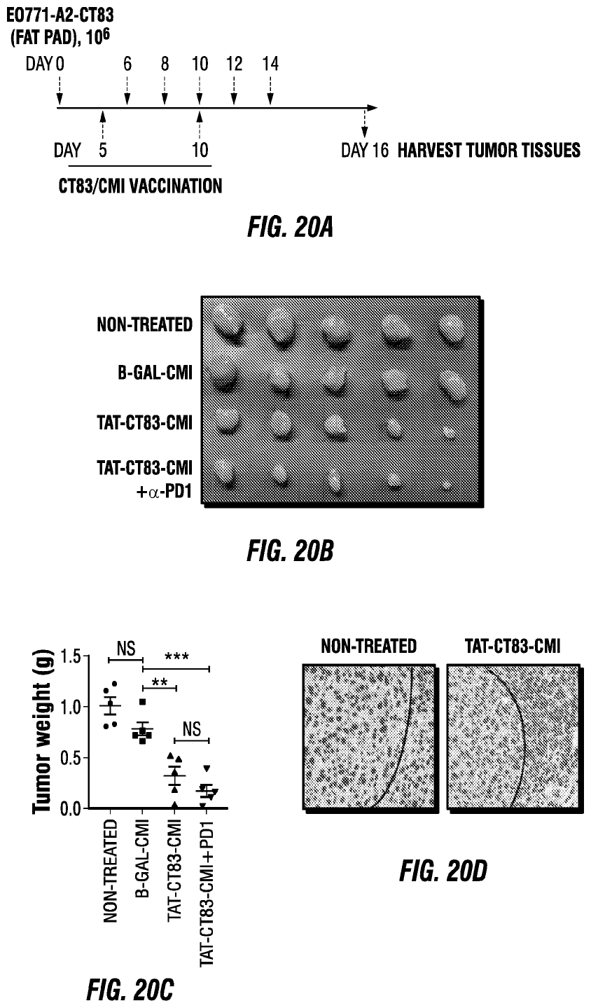

To test this possibility, a series of TAT-linked CT83 peptides containing potential HLA-A2 binging motifs was synthesized (Table 4). Using in vivo immunization of HLA-A2 transgenic mice, it was shown that self-assembled TAT-CT83 peptide nanoparticles with CMI generated strong T cell response against CT83-A2 peptides (FIG. 19A, FIG. 19B, FIG. 19C, FIG. 19D, and FIG. 20A-FIG. 20D). HLA-DR13 and HLA-DP4 restricted T cells were generated after in vitro peptide stimulation.

To determine whether TAT-CT83-CMI could generate potent antitumor immunity, TAT-CT83 peptide vaccines were prepared by mixing 100 μg TAT-CT83 peptide mixture (containing equal amount of TAT-CT83-A2-1, -5, -6 and -7, see Table 4 below), 20 μg CpG, 4 μg MPLA and 10 μg poly(I:C)) under sonication for each mouse. Experimental design using HLA-A2 transgenic mice is shown in FIG. 20A. It was shown that TAT-CT83-CMI vaccine could strongly induce potent antitumor immunity against murine breast cancer E0771/A2/CT83 cells (FIG. 20B and FIG. 20C). Furthermore, such an antitumor immunity could be further enhanced by anti-PD-1 blockade therapy (FIG. 20B and FIG. 20C). Importantly, vaccine-induced T cells infiltrated into tumor tissues, compared with mice without vaccines (FIG. 20D).

TABLE 4
TAT-LINKED CT83 PEPTIDES

| Peptide Names | TAT-Linked Peptides |
|---|---|
| TAT-CT83 A2-1 | YGRKKRRQRRRKLVELEHTL |
| | (SEQ ID NO: 18) |
| TAT-CT83 A2-2 | YGRKKRRQRRRLLASSILCA |
| | (SEQ ID NO: 19) |
| TAT-CT83 A2-3 | YGRKKRRQRRRYLLLASSIL |
| | (SEQ ID NO: 20) |
| TAT-CT83 A2-4 | YGRKKRRQRRRRILVNLSMV |
| | (SEQ ID NO: 21) |
| CT83-DP4-TAT | SILCALIVFWKYRRFQRNYGRKK |
| | (SEQ ID NO: 22) |
| CT83-LP1₁₀₋₃₁ | SILCALIVFWKYRRFQRNTGEM |
| | (SEQ ID NO: 23) |
| CT83-LP2₆₆₋₈₇ | ILNNFPHSIARQKRILVNLSMV |
| | (SEQ ID NO: 24) |
| TAT-CT-83-A2-5 | YGRKKRRQRRRKLVELEHTLLSKG |
| | (SEQ ID NO: 25) |
| TAT-CT-83-A2-6 | YGRKKRRQRRRKLVELEHTLLS |
| | (SEQ ID NO: 26) |
| TAT-CT-83-A2-7 | YGRKKRRQRRRKLVELEHTLL |
| | (SEQ ID NO: 27) |
| TAT-CT-83-A2-8 | YGRKKRRQRRRILNNFPHSI |
| | (SEQ ID NO: 28) |

### 1. TAT-ESO-CMI Generate Strong Antitumor Immunity in Breast Cancer

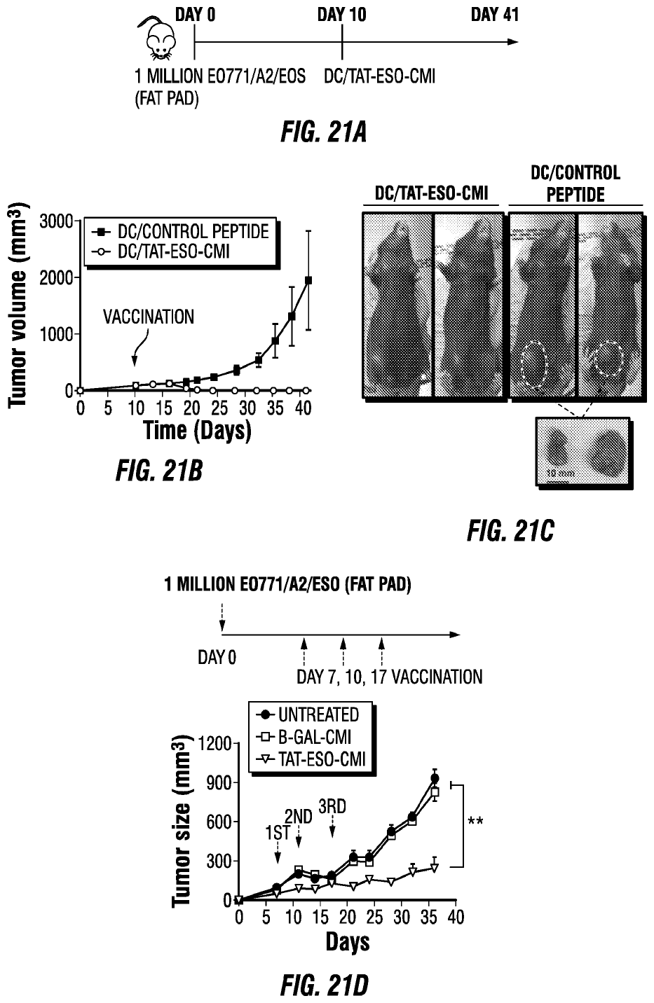

Similarly, experiments were performed using E0771/A2-ESO breast cancer cells in HLA-A2 Tg mice. One vaccination of DC/TAT-ESO-CMI was found at day 10 post tumor injection (1×10⁶ cells/mouse) that resulted in complete inhibition of tumor growth, compared with a control-treated group (FIG. 21A, FIG. 21B, and FIG. 21C). Furthermore, it was shown that vaccination with TAT-ESO-CMI without DC also generated potent antitumor immunity and inhibited tumor cell growth in a therapeutic model (FIG. 21D).

2. Combination Therapy of TAT-TRP2-CMI or TAT-ESO-CMI Vaccine with Anti-PD-1 Blockade:

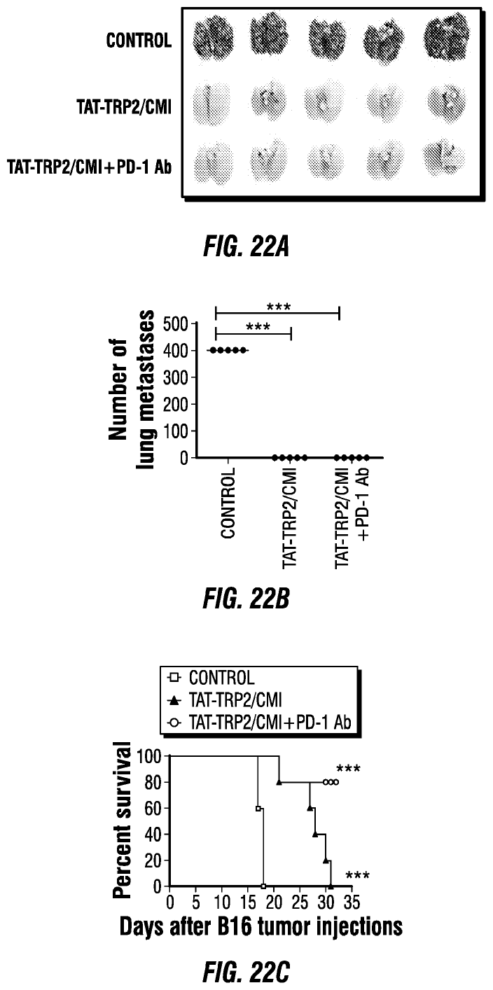

To test whether SAPNANO vaccines could be combined with immune checkpoint therapy, it was shown that TAT-TRP-2-CMI SAPNANO vaccination plus anti-PD-1 therapy could further enhance antitumor immunity and prolong mouse survival compared with TAT-TRP-2-CMI SAPNANO alone (FIG. 22A, FIG. 22B and FIG. 22C). In particular, TAT-TRP2-CMI plus anti-PD-1 markedly prolonged mouse survival (FIG. 22C).

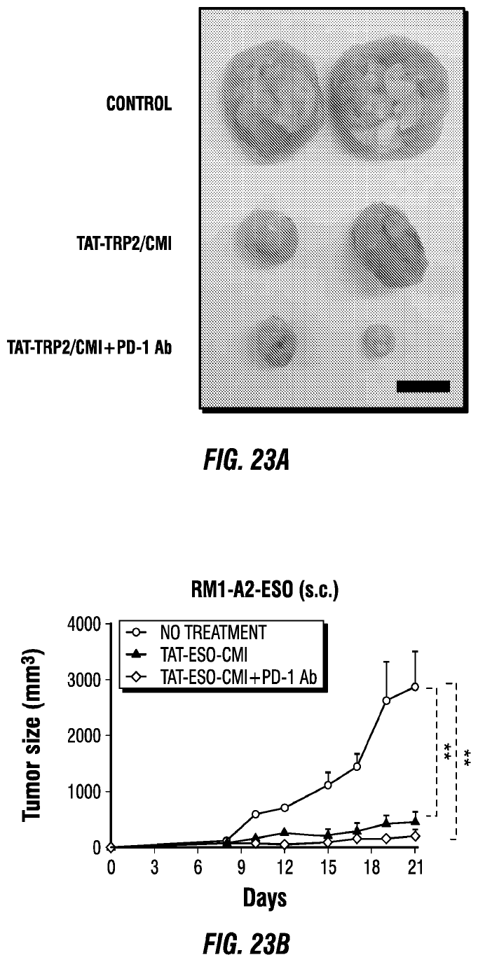

To further test this concept, RM1-A2-ESO tumor-bearing HLA-A2 transgenic mice were treated with TAT-ESO-CMI vaccination alone or combined with anti-PD-1 therapy (FIG. 23A and FIG. 23B). It was shown that SAPNANO vaccine alone markedly inhibited tumor growth (FIG. 22A and FIG. 22B). SAPNANO vaccines-induced antitumor immunity could be further enhanced by anti-PD-1 blockade therapy (FIG. 23A and FIG. 23B).

3. Combination Therapy of TAT-ESO-CMI Vaccine with TCR-T Cell Immunotherapy

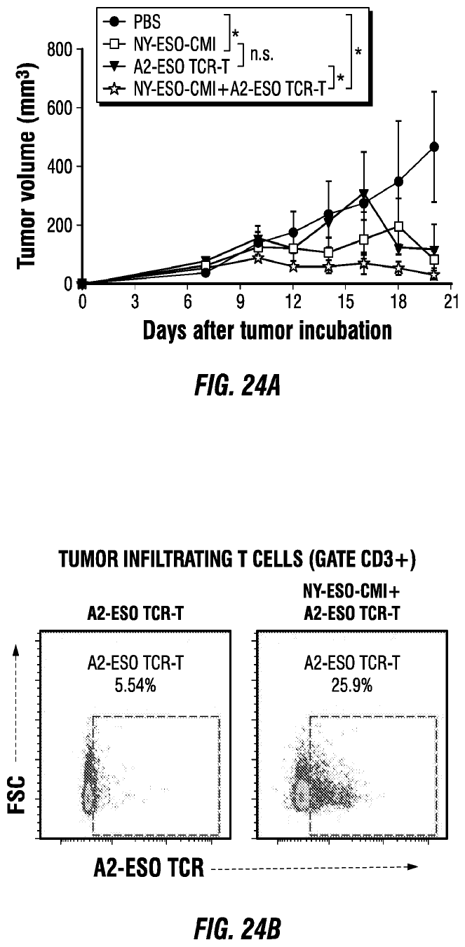

To test whether our novel SAPNANO vaccine could boost A2-ESO TCR-T cell-mediated immunity against breast cancer, an experiment was performed using E0771/A2-ESO tumor cells, and found that adoptive transfer of A2-ESO TCR-T cells followed by TAT-ESO-CMI vaccine inhibited E0771/A2-ESO tumor growth better than either alone (FIG. 24A). Notably, TAT-ESO-CMI vaccine induces stronger antitumor immunity than adoptive transfer of TCR-T cells (FIG. 24A). Consistently, TAT-ESO-CMI vaccine expanded tumor-infiltrating A2-ESO TCR-T cells (25.9%) compared with A2-ESO TCR-T cells alone (5.5%) (FIG. 24B). These results suggest that A2-ESO TCR-T cells could be expanded in vivo by the TAT-ESO-CMI vaccine.

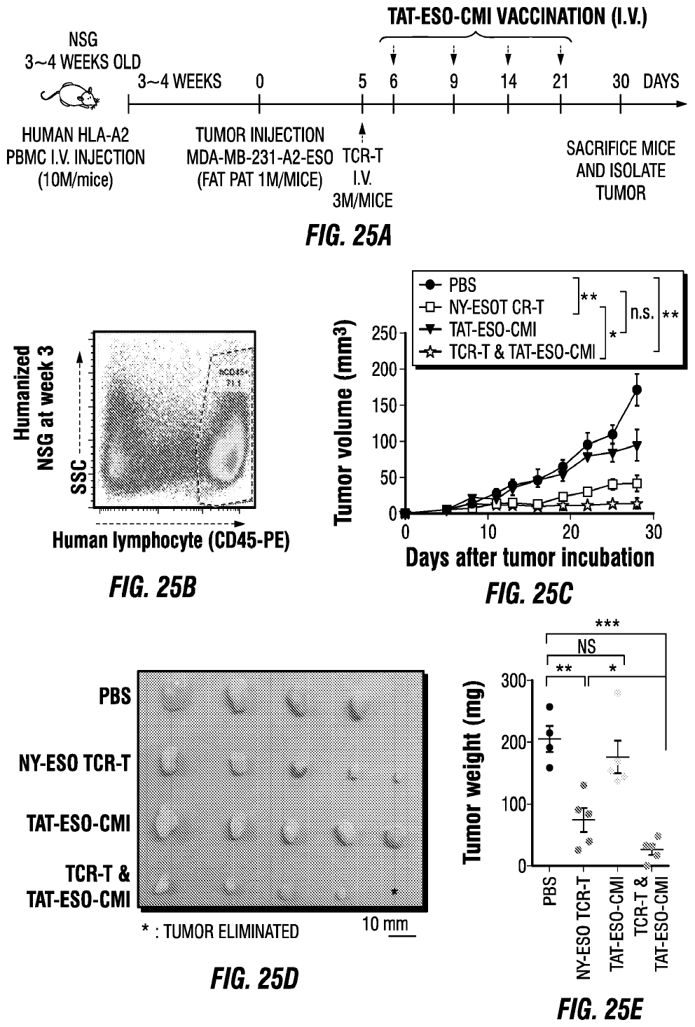

To further test this combined therapy in humanized mice, human PBMCs were injected into NSG mice for reconstituting human immune system for 3-4 weeks. These humanized NSG mice were then injected with MDA-MB-231-A2-ESO tumor cells, followed by SAPNANO vaccines, NY-ESO-1 TCR-T cell therapy, or both. Although the TAT-ESO-CMI vaccine alone did not significantly inhibit the tumor growth due to limited immune cells such as T cells, and DC after immune reconstitution, TAT-ESO-CMI vaccine could be combined with ESO-specific TCR-engineered T cell therapy to generate stronger anti-tumor effect compared with TCR-T cell alone group in MDA-MB-231-A2-ESO breast cancer model than either alone (FIG. 25A, FIG. 25B, FIG. 25C, FIG. 25D, and FIG. 25E).

### REFERENCES

The following references, to the extent that they provide exemplary procedural or other details supplementary to those set forth herein, are specifically incorporated herein in their entirety by express reference thereto:
AKIRA, S, and TAKEDA, K, “Toll-like receptor signalling,” Nat. Rev. Immunol., 4(7):499-511 (July 2004).
ALTSCHUL, S F et al., “Gapped BLAST and PSI-BLAST: a new generation of protein database search programs,” Nucl. Acids Res., 25(17):3389-3402 (1997).
BAGCCHI, S, “Pembrolizumab for treatment of refractory melanoma,” Lancet Oncol., 15(10):e419 (September 2014).
BECHARA, C, and SAGAN, S, “Cell-penetrating peptides: 20 years later, where do we stand?” FEBS Lett., 587(12):1693-1702 (May 2013).
BENDER, E, “Cancer immunotherapy,” Nature, 552:S61 (December 2017).
BLANDER, J M, and MEDZHITOV, R, “On regulation of phagosome maturation and antigen presentation,” Nat. Immunol., 7(10):1029-1035 (October 2006).
BLANDER, J M, and MEDZHITOV, R, “Toll-dependent selection of microbial antigens for presentation by dendritic cells,” Nature, 440(7085):808-812 (February 2006).
BODANSKY, M E et al., Peptide Synthesis. John Wiley & Sons Second Edition (1976).
CHEN et al., “Identification of human tumor antigens by serological expression cloning (SEREX),” In Principles and Practice of Biologic Therapy of Cancer, 3rd Ed. Edited by S. A. Rosenberg, Lippincott, Williams & Wilkins, Philadelphia, Pa., pp. 557-570 (2000).
COLLINS, L et al., “Self-assembly of peptides into spherical nanoparticles for delivery of hydrophilic moieties to the cytosol,” ACS Nano, 4(5):2856-2864 (2010).
CUI, J et al., “NLRC5 negatively regulates the NF-kappaB and type I interferon signaling pathways,” Cell., 141(3):483-496 (April 2010).
CUI, J et al., “NLRP4 negatively regulates type I interferon signaling by targeting the kinase TBK1 for degradation via the ubiquitin ligase DTX4,” Nat. Immunol., 13(4):387-395 (March 2012).
DI LORENZO, G et al., “Immunotherapy for the treatment of prostate cancer,” Nat. Rev. Clin. Oncol., 8(9):551-561 (May 2011).
DOLL, et al., “Design and optimization of peptide nanoparticles,” J. Nanobiotechnol., 13:73 (2015).
ELLIOTT, G, and O′HARE, P, “Intercellular trafficking and protein delivery by a herpesvirus structural protein,” Cell., 88(2):223-233 (January 1997).
FAWELL, S et al., “Tat-mediated delivery of heterologous proteins into cells,” Proc. Nat'l. Acad. Sci. USA, 91(2):664-668 (January 1994).
FRANKEL, A D, and PABO, C O, “Cellular uptake of the tat protein from human immunodeficiency virus,” Cell., 55(6):1189-1193 (December 1988).
FUKUYAMA, T et al., “Identification of a new cancer/germline gene, KK-LC-1, encoding an antigen recognized by autologous CTL induced on human lung adenocarcinoma,” Cancer Res., 66:4922-4928 (May 2006).
GRIBSKOV, M, and BURGESS, R R, “Sigma factors from E. coli, B. subtilis, phage SP01, and phage T4 are homologous proteins,” Nucleic Acids Res., 14(16):6745-6763 (August 1986).
GUNGOR, B et al., “CpG ODN nanorings induce IFNalpha from plasmacytoid dendritic cells and demonstrate potent vaccine adjuvant activity,” Sci. Transl. Med., 6(235):235ra61 (May 2014).
HALE, W G, and MARGHAM, J P, “HARPER COLLINS DICTIONARY OF BIOLOGY,” HarperPerennial, New York (1991).
HAN et al., “Hierarchical assembly of diphenylalanine into dendritic nanoarchitectures,” Colloids Surf B Biointerfaces, 79:440-445 (2010).
HANSEN, M et al., “Predicting cell-penetrating peptides,” Adv. Drug Deliv. Rev., 60(4-5):572-579 (October 2007).
HARDMAN, J G, and LIMBIRD, L E, (Eds.), “GOODMAN AND GILMAN'S THE PHARMACOLOGICAL BASIS OF THERAPEUTICS” 10^{th} Edition, McGraw-Hill, New York (2001).
HEITZ, F et al., “Twenty years of cell-penetrating peptides: From molecular mechanisms to therapeutics,” Br. J. Pharmacol., 157(2):195-206 (March 2009).
HODI, F S et al., “Improved survival with ipilimumab in patients with metastatic melanoma,” N. Engl. J. Med., 363(8):711-723 (June 2010).
HUANG, Y W et al., “Delivery of nucleic acids and nanomaterials by cell-penetrating peptides: opportunities and challenges,” Biomed. Res. Int., 2015:834079 (March 2015).
IWASAKI, A, and MEDZHITOV, R, “Toll-like receptor control of the adaptive immune responses,” Nat. Immunol., 5(10):987-995 (October 2004).
JOHNSON, L A, and JUNE, C H, “Driving gene-engineered T cell immunotherapy of cancer,” Cell Res., 27(1):38-58 (December 2016).
KANTOFF, P W et al., “Sipuleucel-T immunotherapy for castration-resistant prostate cancer,” N. Engl. J. Med., 363(5):411-422 (July 2010).
KIM, D T et al., “Introduction of soluble proteins into the MEW class I pathway by conjugation to an HIV tat peptide,” J. Immunol., 159(4):1666-1668 (August 1997).
KOROLKOV et al., “Surface mediated L-phenylalanyl-L-phenylalanine assembly into large dendritic structures,” Faraday Discuss., 166:257-267 (2013).
KWON, E D et al., “Ipilimumab versus placebo after radiotherapy in patients with metastatic castration-resistant prostate cancer that had progressed after docetaxel chemotherapy (CA184-043): a multicentre, randomised, double-blind, phase 3 trial,” Lancet Oncol., 15(7):700-712 (May 2014).
KYTE, J, and DOOLITTLE, R F, “A simple method for displaying the hydropathic character of a protein,” J. Mol. Biol., 157(1):105-132 (1982).
LESTERHUIS, W et al., “Cancer immunotherapy—revisited,” Nat. Rev. Drug Discov., 10(8):591-600 (August 2011).
LIN, Y Z et al., “Inhibition of nuclear translocation of transcription factor NF-kappa B by a synthetic peptide containing a cell membrane-permeable motif and nuclear localization sequence,” J. Biol. Chem., 270(24):14255-14258 (June 1995).
LINDGREN, M et al., “Cell-penetrating peptides,” Trends Pharmacol. Sci., 21(3):99-103 (March 2000).
MARGUS, H et al., “Characteristics of cell-penetrating peptide/nucleic acid nanoparticles,” Mol. Pharm., 13(1):172-179 (December 2015).
MCCARTHY, H O et al., “Development and characterization of self-assembling nanoparticles using a bio-inspired amphipathic peptide for gene delivery,” J. Control Release, 189:141-149 (2014).
MEINHOFER, J, Hormonal Proteins and Peptides. Academic Press, New York. 2:46. (1983).
MELERO, I et al., “Therapeutic vaccines for cancer: an overview of clinical trials,” Nat. Rev. Clin. Oncol., 11(9):509-524 (July 2014).
MORRIS, M C et al., “Cell-penetrating peptides: from molecular mechanisms to therapeutics,” Biol. Cell., 100(4):201-217 (April 2008).
MUMCUOGLU, D. et al., “Oligonucleotide delivery with cell surface binding and cell penetrating peptide amphiphile nanospheres,” Mol. Pharm., 12(5):1584-1591 (2015).
NANDA, R et al., “Pembrolizumab in Patients With Advanced Triple-Negative Breast Cancer: Phase Ib KEYNOTE-012 Study,” J. Clin. Oncol., 34(21):2460-2467 (May 2016).
NEEDLEMAN, S B and WUNSCH, C D, “A general method applicable to the search for similarities in the amino acid sequence of two proteins,” J. Mol. Biol., 48(3):443-453 (1970).
PALUCKA, K, and BANCHEREAU, J, “Dendritic-cell-based therapeutic cancer vaccines,” Immunity, 39(1):38-48 (July 2013).
PAN, Z Z et al., “Nuclear-targeting TAT-PEG-Asp8-doxorubicin polymeric nanoassembly to overcome drug-resistant colon cancer,” 37(8):1110-1120 (2016).
PARET, C et al., “CXorf61 is a target for T cell based immunotherapy of triple-negative breast cancer,” Oncotarget., 6:25356-25367 (2015).
PHELAN, A et al., “Intercellular delivery of functional p53 by the herpesvirus protein VP22,” Nat. Biotechnol., 16(5):440-443 (May 1998).
RENKVIST et al., A listing of human tumor antigens recognized by T cells. Cancer Immunol. Immunother., 50:3-15. (2001).
ROJAS, M et al., “Genetic engineering of proteins with cell membrane permeability,” Nat. Biotechnol., 16(4):370-375 (April 1998).
ROSENBERG, S A, “Cell transfer immunotherapy for metastatic solid cancer-what clinicians need to know,” Nat. Rev. Clin. Oncol., 8(10):577-585 (August 2011).
ROSENBERG, S A et al., “Cancer immunotherapy: moving beyond current vaccines,” Nat. Med., 10(9):909-915 (September 2004).
SADELAIN, M et al., “Therapeutic T-cell engineering,” Nature, 545(7655):423-431 (May 2017).
SAHIN et al., “Serological identification of human tumor antigens,” Curr. Opin. Immunol., 9:709-716 (1997).
SCHRODER, E, The Peptides. Academic Press, New York (1965).
SCHROEDER, U et al., “Peptide nanoparticles serve as a powerful platform for the immunogenic display of poorly antigenic actin determinants,” J. Mol. Biol., 386(5):1368-1381 (2009).
SCHWARZE, S R et al., “In vivo protein transduction: delivery of a biologically active protein into the mouse,” Science, 285(5433):1569-1572 (September 1999).
SHARMA, P et al., “Primary, adaptive, and acquired resistance to cancer immunotherapy,” Cell., 168(4):707-723 (February 2017).
SINGLETON, P and SAINSBURY, D, “DICTIONARY OF MICROBIOLOGY AND MOLECULAR BIOLOGY,” 2^{nd} Ed., John Wiley and Sons, New York (1987).
SONPAVDE, G et al., “HLA-restricted NY-ESO-1 peptide immunotherapy for metastatic castration resistant prostate cancer,” Invest. New Drugs, 32(2):235-242 (April 2013).
STEWARD, J M, and YOUNG, J D, Solid Phase Peptide Synthesis, W.H. Freeman & Co., San Francisco (1969).
Wan, J et al., “SVRMHC prediction server for MHC-binding peptides,” BMC Bioinformatics, 7:463 (2006).
WAN, Y Y et al., “The kinase TAK1 integrates antigen and cytokine receptor signaling for T cell development, survival and function,” Nature Immunol., 7:851-858 (2006).
WANG, H Y et al., “Induction of CD4+ T cell-dependent antitumor immunity by TAT-mediated tumor antigen delivery into dendritic cells,” J. Clinical Invest., 109(11):1463-1470 (June 2002).
WANG, R F, and WANG, H Y, “Immune targets and neoantigens for cancer immunotherapy and precision medicine,” Cell Res., 27(1):11-37 (December 2016).
WANG, R F, and WANG, H Y, “Enhancement of antitumor immunity by prolonging antigen presentation on dendritic cells,” Nat. Biotechnol., 20(2):149-154 (February 2002).
XIA, X et al., “NLRX1 negatively regulates TLR-induced NF-κB signaling by targeting TRAF6 and IKK,” Immunity, 34(6):843-853 (June 2011).
XIA, X et al., “Porous silicon microparticle potentiates anti-tumor immunity by enhancing cross-presentation and inducing type I interferon response,” Cell. Rep., 11(6):957-966 (April 2015).
YAN et al., “Self-assembly and application of diphenylalanine-based nanostructures,” Chem. Soc. Rev., 39:1877-1890 (2010).
Y U, X et al., “Cross-Regulation of Two Type I Interferon Signaling Pathways in Plasmacytoid Dendritic Cells Controls Anti-malaria Immunity and Host Mortality,” Immunity, 45(5):1093-1107 (October 2016).
ZHU, M et al., “Co-delivery of tumor antigen and dual toll-like receptor ligands into dendritic cell by silicon microparticle enables efficient immunotherapy against melanoma,” J. Control. Release., 272:72-82 (January 2018).
ZOHRABE, T, and HABIBI, N, “Dendritic Peptide Nanostructures Formed from Self-Assembly of Di-L-Phenylalanine Extracted from Alzheimer's β-Amyloid Poly Peptides: Insights into their Assembly Process,” Int. J. Pep. Res. Therapeut., 21:423-431 (2015).

It should be understood that the examples and embodiments described herein are for illustrative purposes only and that various modifications or changes in light thereof will be suggested to persons skilled in the art and are to be included within the spirit and purview of this application and the scope of the appended claims. All references, including publications, patent applications and patents, cited herein are hereby incorporated by reference to the same extent as if each reference was individually and specifically indicated to be incorporated by reference and was set forth in its entirety herein. Recitation of ranges of values herein are merely intended to serve as a shorthand method of referring individually to each separate value falling within the range, unless otherwise indicated herein, and each separate value is incorporated into the specification as if it were individually recited herein.

The description herein of any aspect or embodiment of the invention using terms such as “comprising”, “having”, “including” or “containing” with reference to an element or elements is intended to provide support for a similar aspect or embodiment of the invention that “consists of”, “consists essentially of”, or “substantially comprises” that particular element or elements, unless otherwise stated or clearly contradicted by context (e.g., a composition described herein as comprising a particular element should be understood as also describing a composition consisting of that element, unless otherwise stated or clearly contradicted by context).

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and methods and in the steps or in the sequence of steps of the method described herein without departing from the concept, spirit and scope of the invention. More specifically, it will be apparent that certain agents that are chemically and/or physiologically related may be substituted for the agents described herein while the same or similar results would be achieved.

All such similar substitutes and modifications apparent to those skilled in the art are deemed to be within the spirit, scope and concept of the invention as defined by the appended claims.

What is claimed is:

1. A composition comprising:
(a) a population of nanoparticles, self-assembling at neutral pH, and comprised of a plurality of cationic cell-penetrating peptides covalently linked to a hydrophobic therapeutic peptide ligand;
(b) a pharmaceutically-acceptable buffer, diluent, carrier, or vehicle.

2. The composition in accordance with claim 1, wherein the nanoparticles further comprise a negatively-charged molecule such as mRNA, siRNA, dsRNA, RNA, DNA, or any combination thereof, or a hydrophobic peptide such as MPLA.

3. The composition in accordance with claim 1 or claim 2, further comprising an amphiphobic or amphipathic peptide, such as CPP-TAT covalently linked to a therapeutic peptide, such as NY-ESO-1 (SEQ ID NO:1) or TRP-2 (SEQ ID NO:2).

4. The composition in accordance with any preceding claim, adapted and configured for increasing IFN-I expression, when introduced into suitable mammalian cells; preferably for increasing expression of IFN-α4 or IFN-β.

5. The composition in accordance with any preceding claim, comprised within an isolated population of mammalian cells, such as tumor cells or dendritic cells.

6. The composition in accordance with any preceding claim, comprising one or more cationic cell penetrating peptides as disclosed in any one of SEQ ID NO:3 to SEQ ID NO:8 or SEQ ID NO:11 to SEQ ID NO:16.

7. The composition in accordance with any preceding claim, further comprising: (c) a chemotherapeutic agent, an immunomodulating agent, a neuroactive agent, an anti-inflammatory agent, an anti-lipidemic agent, a hormone, a receptor agonist, a receptor antagonist, an anti-infective agent, an antibody, an antigen-binding fragment of an antibody, a ribozyme, a cofactor, a steroid, or any combination thereof.

8. The composition in accordance with claim 7, wherein the chemotherapeutic agent is selected from the group consisting of cyclophosphamide, doxorubicin, 5-fluorouracil, docetaxel, paclitaxel, trastuzumab, methotrexate, epirubicin, cisplatin, carboplatin, vinorelbine, capecitabine, gemcitabine, mitoxantrone, isabepilone, eribulin, lapatinib, carmustine, a nitrogen mustard, a sulfur mustard, a platin tetranitrate, vinblastine, etoposide, camptothecin, and any combination thereof.

9. The composition in accordance with any preceding claim, further comprising an adjuvant.

10. The composition in accordance with any preceding claim, further comprising an antigen, an antigenic polypeptide, or an antigenic peptide fragment thereof.

11. The composition in accordance with any preceding claim, 1) formulated with a population of liposomes, nanoparticles, or microparticles; or 2) admixed with one or more surfactants, niosomes, ethosomes, transferosomes, phospholipids, or sphingosomes.

12. The composition in accordance with any preceding claim, admixed with one or more pharmaceutically-acceptable carriers, buffers, diluents, vehicles, or excipients.

13. The composition in accordance with any preceding claim, formulated for systemic administration to a mammal, and preferably, for intravenous administration to a human.

14. The composition in accordance with any preceding claim, adapted and configured as part of a therapeutic kit that comprises the composition, and at least a first set of instructions for administration of the composition to a human in need thereof.

15. The composition in accordance with any preceding claim, for use in therapy, prophylaxis, or amelioration of one or more symptoms of a mammalian disease, disorder, dysfunction, deficiency, defect, trauma, injury, or abnormal condition.

16. The composition in accordance with any preceding claim, for use in the therapy, prophylaxis, or amelioration of one or more symptoms of human cancer or infection.

17. An isolated population of mammalian cells comprising the composition in accordance with any preceding claim.

18. The isolated population of mammalian cells in accordance with claim 17, characterized as human dendritic cells.

19. Use of a composition in accordance with any one of claims 1 to 16, in the manufacture of a medicament for treating or ameliorating at least one symptom of a cancer or an infection in a mammalian subject.

20. Use in accordance with claim 19, wherein the mammalian subject is a human, a non-human primate, a companion animal, an exotic, or a livestock.

21. A kit comprising: 1) a composition in accordance with any one of claims 1 to 16; and 2) instructions for administering the composition to a mammal in need thereof, as part of a regimen for the prevention, diagnosis, treatment, or amelioration of one or more symptoms of a disease, a dysfunction, an abnormal condition, or a trauma in the mammal.

22. A method of treating or ameliorating one or more symptoms of cancer or an infection in an animal in need thereof, the method comprising administering to the animal an effective amount of a composition in accordance with any one of claims 1 to 16, for a time sufficient to treat or ameliorate the one or more symptoms of the cancer or the infection in the animal.

23. The method in accordance with claim 22, wherein the cancer is diagnosed as, or is identified as, a refractory, a metastatic, a relapsed, or a treatment-resistant cancer.

24. The method in accordance with claim 22 or 23, wherein the animal is human.

25. The method in accordance with any one of claims 22 to 24, wherein the composition is administered systemically to the animal, in a single administration, or in a series of multiple administrations over a period of from one or more days, over a period of one or more weeks, or over a period of one or more months or longer.

26. The method in accordance with any one of claims 22 to 25, wherein the composition further comprises a second distinct chemotherapeutic agent, or a second distinct population of self-assembling nanoparticles that comprises a second distinct therapeutic agent.

27. A method of administering a diagnostic, therapeutic, or prophylactic agent to one or more cells, tissues, organs, or systems of a mammalian subject in need thereof, comprising administering to the subject an effective amount of the composition of in accordance with any one of claims 1 to 16.

28. The method in accordance with claim 27, wherein the one or more cells are human dendritic cells.

29. The method in accordance with claim 27, wherein the one or more tissues is tumoral.

30. A method of providing a therapeutic composition to at least one cell, at least one tissue, or at least one organ of a patient in need thereof, comprising administering to a patient in need thereof, an amount of a composition in accordance with any one of claims 1 to 16, or an isolated population of mammalian cells in accordance with claim. 17 or claim 18, and for a time effective to provide the therapeutic composition to at least one cell, at least one tissue, or at least one organ of the patient.
